(19) United States
(12) Patent Application Publication
(10) Pub. No.: US 2017/0340629 A1
(43) Pub. Date: Nov. 30, 2017

(54) MASITINIB COMBINATION FOR USE IN TREATING BREAST CANCER

(71) Applicant: AB SCIENCE, Paris (FR)

(72) Inventors: Alain MOUSSY, Paris (FR); Jean-Pierre KINET, Lexington, MA (US); Colin MANSFIELD, Ecully (FR)

(73) Assignee: AB SCIENCE, Paris (FR)

(21) Appl. No.: 15/536,335

(22) PCT Filed: Dec. 15, 2015
(86) PCT No.: PCT/EP2015/079907

§ 371 Date: Jun. 15, 2017

(30) Foreign Application Priority Data
Dec. 15, 2014 (EP) 14198065.6

(51) Int. Cl. A61K 31/496; A61K 31/7068; C12Q 1/68; A61K 45/06

(52) U.S. Cl. CPC A61K 31/496; C12Q 1/6886; A61K 31/7068; A61K 45/06; C12Q 2600/106; C12Q 2600/158

(57) ABSTRACT

Disclosed is a method for treating breast cancer in a subject in need thereof, including administering to the subject a therapeutically effective amount of a tyrosine kinase inhibitor or a pharmaceutically acceptable salt or solvate thereof, optionally in combination with a therapeutically effective amount of a chemotherapeutic agent.

### FIELD OF INVENTION

The present invention relates to the treatment of breast cancer. More specifically, the present invention relates to the treatment of breast cancer using masitinib.

### BACKGROUND OF INVENTION

Breast cancer (BC) is the most common female cancer. More than 1 million women worldwide are affected by this diagnosis and 400 000 patients die due to the disease every year. Implementation of mammography screening as well as improvement of adjuvant systemic treatment and a decrease in hormone replacement therapy use have resulted in a decrease in both BC incidence and particularly mortality in developed countries over the past 5 years; worldwide, however, the incidence of BC is nevertheless increasing.

4 stages of breast cancer may be defined according to the tumor, node and metastasis (TNM) classification:
stage 0 (carcinoma in situ) encompasses three different situations:
   Ductal carcinoma in situ (DCIS): noninvasive condition in which abnormal cells are found in the lining of a breast duct.
   Lobular carcinoma in situ (LCIS): abnormal cells are found in the lobules of the breast.
   Paget disease of the nipple: abnormal cells are found in the nipple only.
stage I comprises two subgroups, namely stages IA and IB:
   In stage IA, the tumor is 2 centimeters or smaller. Cancer has not spread outside the breast.
   In stage IB, small clusters of breast cancer cells (larger than 0.2 millimeter but not larger than 2 millimeters) are found in the lymph nodes and either no tumor is found in the breast; or the tumor is 2 centimeters or smaller.
stage II also comprises two subgroups, stages IIA and IIB:
   According to the United States National Cancer Institute definition of stage IIA breast cancer, two situations may occur: either (i) no tumor is found in the breast or the tumor is 2 centimeters or smaller. Moreover, cancer (larger than 2 millimeters) is found in 1 to 3 axillary lymph nodes or in the lymph nodes near the breastbone; or (ii) the tumor is larger than 2 centimeters but not larger than 5 centimeters, and cancer has not spread to the lymph nodes.
   In stage IIB, three situations may occur, which are characterized by the fact that the tumor is:
      i. larger than 2 centimeters but not larger than 5 centimeters. Small clusters of breast cancer cells (larger than 0.2 millimeter but not larger than 2 millimeters) are found in the lymph nodes; or
      ii. larger than 2 centimeters but not larger than 5 centimeters. Cancer has spread to 1 to 3 axillary lymph nodes or to the lymph nodes near the breastbone (found during a sentinel lymph node biopsy); or
      iii. larger than 5 centimeters. Cancer has not spread to the lymph nodes.
stage III is characterized by the fact that cancer has locally spread, and may refer to three different substages:
   According to the United States National Cancer Institute definition of stage IIIA breast cancer, encompasses three situations, which are defined as follows:
      no tumor is found in the breast or the tumor may be any size. Cancer is found in 4 to 9 axillary lymph nodes or in the lymph nodes near the breastbone; or
      the tumor is larger than 5 centimeters. Small clusters of breast cancer cells (larger than 0.2 millimeter but not larger than 2 millimeters) are found in the lymph nodes; or
      the tumor is larger than 5 centimeters. Cancer has spread to 1 to 3 axillary lymph nodes or to the lymph nodes near the breastbone.
   In stage IIIB, the tumor may be any size and cancer has spread to the chest wall and/or to the skin of the breast and caused swelling or an ulcer. Also, cancer may have spread to up to 9 axillary lymph nodes; or to the lymph nodes near the breastbone.
   In stage IIIC, no tumor is found in the breast or the tumor may be any size. Cancer may have spread to the skin of the breast and caused swelling or an ulcer and/or has spread to the chest wall. Also, cancer has spread to (i) 10 or more axillary lymph nodes; or (ii) lymph nodes above or below the collarbone; or (iii) axillary lymph nodes and lymph nodes near the breastbone. For treatment, stage IIIC breast cancer is divided into operable and inoperable stage IIIC. Stage IIIC cancer may be inoperable in particular in cases where the cancer has spread to lymph nodes above the collarbone and near the neck on the same side of the body as the affected breast.
stage IV (metastatic breast cancer): cancer has spread to other organs of the body (most often the bones, lungs, liver or brain).

Among these stages of breast cancer, stage III (corresponding to a stage wherein cancer has spread locally to the area of the breast, but has not spread to distant organs and tissues) and stage IV (metastatic breast cancer) may also be referred as “advanced breast cancer”. Moreover, advanced breast cancer may also include inflammatory breast cancer, wherein cancer has spread to the skin of the breast, and which may meet the criteria for stages IIIB, IIIC or IV. Hence, because inflammatory breast cancer is either stage III or IV at diagnosis, depending on whether cancer cells have spread only to nearby lymph nodes or to other tissues as well, this type of cancer intrinsically falls under the categorization of advanced breast cancer. Furthermore, the United States National Cancer Institute also states that inflammatory breast tumors are frequently hormone receptor negative, which means that hormone therapies, such as tamoxifen, that interfere with the growth of cancer cells fueled by estrogen may not be effective against these tumors.

Due to the poor prognosis of advanced breast cancer as compared to other stages, there is still a need for agents or combinations of agents with an enhanced therapeutic efficacy on advanced breast cancer.

Inflammatory breast cancer (IBC) is a rapidly progressive tumor, with propensity for metastatic tumor spread, and poor overall survival (OS) compared with non-IBC locally advanced breast cancer. There are currently no treatment options for patients with refractory IBC. Evidence suggests that IBC is phenotypically distinct from other types of breast cancer, for example, it is distinguished by overexpression of biomarkers; however to date differences on the molecular level remain to be elucidated. In research by Masuda and colleagues (Masuda H et al. Breast Cancer Res. 2013 Nov. 25; 15(6):R112) it was shown that although clinical characteristics differ significantly between IBC and non-IBC patients with triple negative breast cancer (TNBC), these patient groups had the same mRNA expression profiles and could not therefore be distinguished via triple-negative subtype. These findings led to the conclusion that the differences in triple negative-IBC and triple negative-non-IBC are either due to very subtle molecular difference within the tumor or differences in the tumor microenvironmental, i.e. independent of the tumor itself. Other research by Zell and colleagues (Zell J A, et al. Breast Cancer Res. 2009; 11(1):R9) showed that despite an association with advanced tumor stage, HER2+ (human epidermal growth factor receptor 2) status was not an independent adverse prognostic factor for survival among IBC patient cases. These findings lead to the conclusion that IBC's poor survival is probably unrelated to HER2 status and that other, unknown, clinical factors may explain the poor overall survival for IBC.

One possible explanation for these observations is the involvement of mast cells and tumor associated macrophages in supporting tumoral pathogenesis via modulating the tumor microenvironment and release of pro-inflammatory, pro-tumoral cytokines.

There are currently no treatment options for patients with refractory IBC. Kaufman and colleagues reported findings from a phase II study of lapatinib (1500 mg/day) in 126 patients with relapsed or refractory inflammatory breast cancer (Kaufman B, et al. Lancet Oncol. 2009 June; 10(6):581-8). Lapatinib is an inhibitor of epidermal growth factor receptors. Median overall survival was 11.2 months.

In particular, advanced triple-negative breast cancer (TNBC) has very poor overall survival. TNBC is a relatively new subset of breast cancer and affects around 10 to 20% of women. It is characterized by the absence of the estrogen-receptor (ER) and of the progesterone-receptor (PR), and by the absence of overexpression of the human epidermal growth factor receptor type 2 (HER2). It has been reported that up to 40% of inflammatory breast cancer cases are also TNBC. Metastatic TNBC is a particularly aggressive subtype of breast cancer marked by higher rates of visceral and central nervous system metastases, and poorer disease-specific survival than hormone receptor-positive subtypes. Patients with TNBC treated with preoperative chemotherapy showed higher rates of pathological complete response than patients with hormone receptor-positive breast cancer. However, when residual disease exists, the prognosis is worse in TNBC than other subtypes of breast cancer with residual disease after chemotherapy, and the median survival is of approximately 1 year.

Currently there are no targeted therapies approved by the FDA or EMA specifically for treatment of TNBC. Because hormones (i.e. hormone receptors and HER2) do not support the growth of TNBC, this cancer is unlikely to respond to endocrine therapies (e.g. tamoxifen) or medications that target HER2 (e.g. trastuzumab). TNBC patients therefore generally receive traditional chemotherapy as standard of care treatment, similar to patients with high-risk or metastatic breast cancer.

Therefore, there is still a need for therapeutic options for treating advanced breast cancer, preferably advanced TNBC.

Masitinib mesilate is a novel tyrosine kinase inhibitor part of 2-aminoarylthiazoles derivatives that mainly targets c-Kit, and the angiogenic PDGF receptors but was also found to target the non-receptor tyrosine kinases Lyn and to a lower extent FGFR3 (Dubreuil et al., 2009, PLoS ONE 2009.4(9):e7258).

The Applicant herein surprisingly demonstrates that masitinib potentiates cytotoxic effect of chemotherapies in breast cancer, including gemcitabine, doxorubicin, mitoxantrone, cisplatin, carboplatin, capecitabine and irinotecan. The present invention thus relates to the synergistic combination of masitinib and chemotherapeutic agents for treating breast cancer.

### SUMMARY

The present invention relates to a method for treating breast cancer in a subject in need thereof, comprising administering to said subject a therapeutically effective amount of a tyrosine kinase inhibitor or mast cell inhibitor or a pharmaceutically acceptable salt or solvate thereof, optionally in combination with a therapeutically effective amount of at least one chemotherapeutic agent.

In one embodiment, the tyrosine kinase inhibitor, c-Kit inhibitor or mast cell inhibitor is masitinib or a pharmaceutically acceptable salt or solvate thereof. In one embodiment, the pharmaceutically acceptable salt or solvate of masitinib is masitinib mesilate.

In one embodiment, the tyrosine kinase inhibitor or mast cell inhibitor is an inhibitor of c-Kit, Lyn, Fyn and/or PDGFR α and β. In one embodiment, the tyrosine kinase inhibitor is an inhibitor of c-Kit.

The present invention also relates to a method for treating breast cancer in a subject in need thereof, comprising administering to said subject a therapeutically effective amount of a c-Kit inhibitor or mast cell inhibitor or a pharmaceutically acceptable salt or solvate thereof, in combination with a therapeutically effective amount of at least one chemotherapeutic agent.

In one embodiment, the c-Kit inhibitor or mast cell inhibitor is an inhibitor of c-Kit, Lyn, Fyn and/or PDGFR α and β. In one embodiment, the c-Kit inhibitor or mast cell inhibitor is masitinib or a pharmaceutically acceptable salt or solvate thereof. In one embodiment, the pharmaceutically acceptable salt or solvate of masitinib is masitinib mesilate.

In one embodiment, the method of the invention is for improving survival and/or life expectancy of the subject.

In one embodiment, the method of the invention comprises sensitizing to a chemotherapeutic agent or restoring sensitivity to chemotherapy in the subject.

In one embodiment, breast cancer is advanced breast cancer, preferably locally advanced breast cancer or metastatic breast cancer. In one embodiment, breast cancer is triple-negative breast cancer (TNBC), preferably advanced TNBC, more preferably locally advanced TNBC or metastatic TNBC.

In one embodiment, breast cancer is inflammatory breast cancer (IBC), preferably advanced IBC, more preferably locally advanced IBC or metastatic IBC. In one embodiment, breast cancer is triple-negative inflammatory breast cancer.

In one embodiment, breast cancer is relapsed or is refractory breast cancer.

In one embodiment, the subject is naïve to anti-breast cancer treatments, or breast cancer has relapsed after at least one anti-breast cancer treatment, or after two or more anti-breast cancer treatments. In one embodiment, said anti-breast cancer treatments include treatment with one or more chemotherapeutic agent, preferably anthracyclines and/or taxanes, surgery or radiotherapy.

In one embodiment, said at least one chemotherapeutic agent is selected from the group consisting of: abitrexate (Methotrexate), abraxane (Paclitaxel Albumin-stabilized Nanoparticle Formulation), ado-Trastuzumab Emtansine, adrucil (Fluorouracil), afinitor (Everolimus), anastrozole, aredia (Pamidronate Disodium), arimidex (Anastrozole), aromasin (Exemestane), carboplatin, capecitabine, cisplatin, Clafen (Cyclophosphamide), Cyclophosphamide, Cytoxan (Cyclophosphamide), Docetaxel, Doxorubicin Hydrochloride, Efudex (Fluorouracil), Ellence (Epirubicin Hydrochloride), Epirubicin Hydrochloride, Eribulin Mesylate, Everolimus, Exemestane, Fareston (Toremifene), Faslodex (Fulvestrant), Femara (Letrozole), Fluoroplex (Fluorouracil), Fluorouracil, Folex (Methotrexate), Folex PFS (Methotrexate), Fulvestrant, Gemcitabine Hydrochloride, Gemzar (Gemcitabine Hydrochloride), Goserelin Acetate, Halaven (Eribulin Mesylate), Herceptin (Trastuzumab), Ixabepilone, Ixempra (Ixabepilone), Kadcyla (Ado-Trastuzumab Emtansine), Lapatinib Ditosylate, Letrozole, Megace (Megestrol Acetate), Megestrol Acetate, Methotrexate, Methotrexate LPF (Methotrexate), Mexate (Methotrexate), Mexate-AQ (Methotrexate), mitoxantrone, Neosar (Cyclophosphamide), Nolvadex (Tamoxifen Citrate), Paclitaxel, Paclitaxel Albumin-stabilized Nanoparticle Formulation, Pamidronate Disodium, Perjeta (Pertuzumab), Pertuzumab, Tamoxifen Citrate, Taxol (Paclitaxel), Taxotere (Docetaxel), Trastuzumab, Toremifene, Tykerb (Lapatinib Ditosylate), Velban (Vinblastine Sulfate), Velsar (Vinblastine Sulfate), Vinblastine Sulfate, Xeloda (Capecitabine), Zoladex (Goserelin Acetate), and Irinotecan.

In one embodiment, said at least one chemotherapeutic agent is selected from anthracyclines, taxanes, platinum based chemotherapeutic agents, antimetabolites, and mixtures thereof.

In one embodiment; said at least one chemotherapeutic agent is selected from anthracyclines. In one embodiment said anthracycline is selected from the group consisting of doxorubicin, epirubicin, mitoxantrone, pixantrone, losoxantrone, and daunorubicin, or any mixtures thereof. In another embodiment said anthracycline is doxorubicin or mitoxantrone.

In one embodiment, breast cancer is locally advanced or metastatic breast cancer, and said at least one chemotherapeutic agent is an antimetabolite, an anthracycline, a taxane, a platinum based chemotherapeutic agent or any mixtures thereof, preferably selected from the group consisting of gemcitabine, carboplatin, capecitabine, doxorubicin and mitoxantrone or any mixtures thereof.

In one embodiment, breast cancer is TNBC and said at least one chemotherapeutic agent is an antimetabolite, an anthracycline, a taxane, a platinum based chemotherapeutic agent or any mixtures thereof, preferably selected from the group consisting of cisplatin, gemcitabine, carboplatin, doxorubicin and mitoxantrone or any combination thereof.

In one embodiment, breast cancer is IBC, and said at least one chemotherapeutic agent is an antimetabolite, an anthracycline, a taxane, a platinum based chemotherapeutic agent or any mixtures thereof, preferably selected from the group consisting of gemcitabine, capecitabine, cisplatin, carboplatin, doxorubicin and mitoxantrone or any mixtures thereof.

In one embodiment, the therapeutically effective amount of said tyrosine kinase inhibitor ranges from about 6 mg/kg/day to about 9 mg/kg/day.

In one embodiment, said tyrosine kinase inhibitor is orally administered.

The present invention also relates to a method for inhibiting tyrosine kinases, preferably selected from the group consisting of c-Kit, LYN, FYN and PDGFR α and β and for inducing an anti-tumoral Th1 immune response, in a breast cancer patient, thereby treating breast cancer, wherein said method comprises administering a therapeutically effective amount of a tyrosine kinase inhibitor, preferably a c-Kit inhibitor, or mast cell inhibitor, preferably masitinib, or a pharmaceutically acceptable salt or solvate thereof in combination with a therapeutically effective amount of a chemotherapeutic agent.

The present invention also relates to a pharmaceutical composition comprising a tyrosine kinase inhibitor, preferably a c-Kit inhibitor, or mast cell inhibitor or a pharmaceutically acceptable salt or solvate thereof and a chemotherapeutic agent, in combination with at least one pharmaceutically acceptable carrier, wherein said tyrosine kinase inhibitor is preferably masitinib mesilate, and wherein said chemotherapeutic agent is preferably selected from cisplatin, gemcitabine, carboplatin, capecitabine, doxorubicin, mitoxantrone and mixtures thereof.

The present invention also relates to a medicament comprising a tyrosine kinase inhibitor, preferably a c-Kit inhibitor, or mast cell inhibitor or a pharmaceutically acceptable salt or solvate thereof and a chemotherapeutic agent, wherein said tyrosine kinase inhibitor is preferably masitinib mesilate, and wherein said chemotherapeutic agent is preferably selected from cisplatin, gemcitabine, carboplatin, capecitabine, doxorubicin, mitoxantrone and mixtures thereof.

The present invention also relates to a kit of part comprising, in a first part, a tyrosine kinase inhibitor, preferably a c-Kit inhibitor, or mast cell inhibitor or a pharmaceutically acceptable salt or solvate thereof, preferably wherein said tyrosine kinase inhibitor is masitinib mesilate, and, in a second part, a chemotherapeutic agent, preferably selected from cisplatin, gemcitabine, carboplatin, capecitabine, doxorubicin, mitoxantrone and mixtures thereof.

The present invention also relates to the pharmaceutical composition as defined hereinabove, the medicament as defined hereinabove or the kit of part as defined hereinabove, for treating breast cancer.

### Definitions

In the present invention, the following terms have the following meanings:
The term “subject” refers to a mammal, preferably a human. In one embodiment, the subject is a man. In another embodiment, the subject is a woman. In one embodiment, a subject may be a “patient”, i.e. a warm-blooded animal, more preferably a human, who/which is awaiting the receipt of, or is receiving medical care or was/is/will be the object of a medical procedure, or is monitored for the development of a breast cancer. In one embodiment, the subject is an adult (for example a subject above the age of 18). In another embodiment, the subject is a child (for example a subject below the age of 18). In one embodiment, the subject is a male. In another embodiment, the subject is a female.
The terms “treating” or “treatment” refers to both therapeutic treatment and prophylactic or preventative measures; wherein the object is to prevent or slow down (lessen) breast cancer. Those in need of treatment include those already with breast cancer as well as those prone to have breast cancer or those in whom breast cancer is to be prevented. A subject is successfully “treated” for breast cancer if, after receiving a therapeutic amount of a tyrosine kinase inhibitor or mast cell inhibitor according to the methods of the present invention, the patient shows observable and/or measurable reduction in or absence of one or more of the following: reduction in the number of pathogenic cells; reduction in the percent of total cells that are pathogenic; and/or relief to some extent, of one or more of the symptoms associated with breast cancer; reduced morbidity and mortality, and improvement in quality of life issues. The above parameters for assessing successful treatment and improvement in the disease are readily measurable by routine procedures familiar to a physician.
The term “therapeutically effective amount” means the level or amount of agent that is aimed at, without causing significant negative or adverse side effects to the target, (1) delaying or preventing the onset of breast cancer; (2) slowing down or stopping the progression, aggravation, or deterioration of one or more symptoms of breast cancer; (3) bringing about ameliorations of the symptoms of breast cancer; (4) reducing the severity or incidence of breast cancer; or (5) curing breast cancer. A therapeutically effective amount may be administered prior to the onset of breast cancer, for a prophylactic or preventive action. Alternatively or additionally, the therapeutically effective amount may be administered after initiation of breast cancer, for a therapeutic action or maintenance of a therapeutic action.
The term “pharmaceutically acceptable carrier or excipient” refers to an excipient or carrier that does not produce an adverse, allergic or other untoward reaction when administered to an animal, preferably a human. It includes any and all solvents, dispersion media, coatings, antibacterial and antifungal agents, isotonic and absorption delaying agents and the like. For human administration, injected preparations should meet sterility, pyrogenicity, general safety and purity standards as required by regulatory offices, such as, for example, FDA Office or EMA.
The term “about” preceding a figure means plus or less 10% of the value of said figure.
As used herein, the term an “aryl group” means a monocyclic or polycyclic-aromatic radical comprising carbon and hydrogen atoms. Examples of suitable aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. An aryl group can be unsubstituted or substituted with one or more substituents. In one embodiment, the aryl group is a monocyclic ring, wherein the ring comprises 6 carbon atoms, referred to herein as “(C6)aryl”.
As used herein, the term “alkyl group” means a saturated straight chain or branched non-cyclic hydrocarbon having from 1 to 10 carbon atoms. Representative saturated straight chain alkyls include methyl, ethyl, n-propyl, n-butyl, n-pentyl, n-hexyl, n-heptyl, n-octyl, n-nonyl and n-decyl; while saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. Alkyl groups included in compounds of this invention may be optionally substituted with one or more substituents.
As used herein, the term “alkoxy” refers to an alkyl group which is attached to another moiety by an oxygen atom. Examples of alkoxy groups include methoxy, isopropoxy, ethoxy, tert-butoxy, and the like. Alkoxy groups may be optionally substituted with one or more substituents.
As used herein, the term “heteroaryl” or like terms means a monocyclic or polycyclic heteroaromatic ring comprising carbon atom ring members and one or more heteroatom ring members (such as, for example, oxygen, sulfur or nitrogen). Typically, a heteroaryl group has from 1 to about 5 heteroatom ring members and from 1 to about 14 carbon atom ring members. Representative heteroaryl groups include pyridyl, 1-oxo-pyridyl, furanyl, benzo[1,3]dioxolyl, benzo[1,4]dioxinyl, thienyl, pyrrolyl, oxazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzo(b)thienyl. A heteroatom may be substituted with a protecting group known to those of ordinary skill in the art, for example, the hydrogen on a nitrogen may be substituted with a tert-butoxycarbonyl group. Heteroaryl groups may be optionally substituted with one or more substituents. In addition, nitrogen or sulfur heteroatom ring members may be oxidized. In one embodiment, the heteroaromatic ring is selected from 5-8 membered monocyclic heteroaryl rings. The point of attachment of a heteroaromatic or heteroaryl ring to another group may be at either a carbon atom or a heteroatom of the heteroaromatic or heteroaryl rings.
The term “heterocycle” as used herein, refers collectively to heterocycloalkyl groups and heteroaryl groups.
As used herein, the term “heterocycloalkyl” means a monocyclic or polycyclic group having at least one heteroatom selected from O, N or S, and which has 2-11 carbon atoms, which may be saturated or unsaturated, but is not aromatic. Examples of heterocycloalkyl groups include (but are not limited to): piperidinyl, piperazinyl, 2-oxopiperazinyl, 2-oxopiperidinyl, 2-oxopyrrolidinyl, 4-piperidonyl, pyrrolidinyl, hydantoinyl, valerolactamyl, oxiranyl, oxetanyl, tetrahydropyranyl, tetrahydrothiopyranyl, tetrahydropyrindinyl, tetrahydropyrimidinyl, tetrahydrothiopyranyl sulfone, tetrahydrothiopyranyl sulfoxide, morpholinyl, thiomorpholinyl, thiomorpholinyl sulfoxide, thiomorpholinyl sulfone, 1,3-dioxolane, tetrahydrofuranyl, dihydrofuranyl-2-one, tetrahydrothienyl, and tetrahydro-1,1-dioxothienyl. Typically, monocyclic heterocycloalkyl groups have 3 to 7 members. Preferred 3 to 7 membered monocyclic heterocycloalkyl groups are those having 5 or 6 ring atoms. A heteroatom may be substituted with a protecting group known to those of ordinary skill in the art, for example, the hydrogen on a nitrogen may be substituted with a tert-butoxycarbonyl group. Furthermore, heterocycloalkyl groups may be optionally substituted with one or more substituents. In addition, the point of attachment of a heterocyclic ring to another group may be at either a carbon atom or a heteroatom of a heterocyclic ring. Only stable isomers of such substituted heterocyclic groups are contemplated in this definition.
As used herein the term “substituent” or “substituted” means that a hydrogen radical on a compound or group is replaced with any desired group that is substantially stable to reaction conditions in an unprotected form or when protected using a protecting group. Examples of preferred substituents are those found in the exemplary compounds and embodiments disclosed herein, as well as halogen (chloro, iodo, bromo, or fluoro); alkyl; alkenyl; alkynyl; hydroxy; alkoxy; nitro; thiol; thioether; imine; cyano; amido; phosphonato; phosphine; carboxyl; thiocarbonyl; sulfonyl; sulfonamide; ketone; aldehyde; ester; oxygen (—O); haloalkyl (e.g., trifluoromethyl); cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g., pyrrolidinyl, piperidinyl, piperazinyl, morpholinyl, or thiazinyl), monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidinyl, benzimidazolyl, benzothiophenyl, or benzofuranyl); amino (primary, secondary, or tertiary); CO₂CH₃; CONH₂; OCH₂CONH₂; NH₂; SO₂NH₂; OCHF₂; CF₃; OCF₃; and such moieties may also be optionally substituted by a fused-ring structure or bridge, for example —OCH₂O—. These substituents may optionally be further substituted with a substituent selected from such groups. In certain embodiments, the term “substituent” or the adjective “substituted” refers to a substituent selected from the group consisting of an alkyl, an alkenyl, an alkynyl, an cycloalkyl, an cycloalkenyl, a heterocycloalkyl, an aryl, a heteroaryl, an aralkyl, a heteraralkyl, a haloalkyl, —C(O)NR₁₁R₁₂, —NR₁₃C(O)R₁₄, a halo, —OR₁₃, cyano, nitro, a haloalkoxy, —C(O)R₁₃, —NR₁₁R₁₂, —SR₁₃, —C(O)OR₁₃, —OC(O)R₁₃, —NR₁₃C(O)NR₁₁R₁₂, —OC(O)NR₁₁R₁₂, —NR₁₃C(O)OR₁₄, —S(O)rR₁₃, —NR₁₃S(O)rR₁₄, —OS(O)rR₁₄, S(O)rNR₁₁R₁₂, —O, —S, and —N—R₁₃, wherein r is 1 or 2; R₁₁ and R₁₂, for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₁₁ and R₁₂ taken together with the nitrogen to which they are attached is optionally substituted heterocycloalkyl or optionally substituted heteroaryl; and R₁₃ and R₁₄ for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl. In certain embodiments, the term “substituent” or the adjective “substituted” refers to a solubilising group.
The term “solubilising group” means any group which can be substantially ionized and that enables the compound to be soluble in a desired solvent, such as, for example, water or water-containing solvent. Furthermore, the solubilising group can be one that increases the compound or complex's lipophilicity. Typically, the solubilising group is selected from alkyl group substituted with one or more heteroatoms such as N, O, S, each optionally substituted with alkyl group substituted independently with alkoxy, amino, alkylamino, dialkylamino, carboxyl, cyano, or substituted with cycloheteroalkyl or heteroaryl, or a phosphate, or a sulfate, or a carboxylic acid. For example, by “solubilising group” it is referred herein to one of the following:
   an alkyl, cycloalkyl, aryl, heretoaryl group comprising either at least one nitrogen or oxygen heteroatom or which group is substituted by at least one amino group or oxo group;
   an amino group which may be a saturated cyclic amino group which may be substituted by a group consisting of alkyl, alkoxycarbonyl, halogen, haloalkyl, hydroxyalkyl, amino, monoalkylamino, dialkylamino, carbamoyl, monoalkylcarbamoyl and dialkylcarbamoyl;
   one of the structures a) to i) shown below, wherein the wavy line and the arrow line correspond to the point of attachment to core structure of formula (A) or (B)

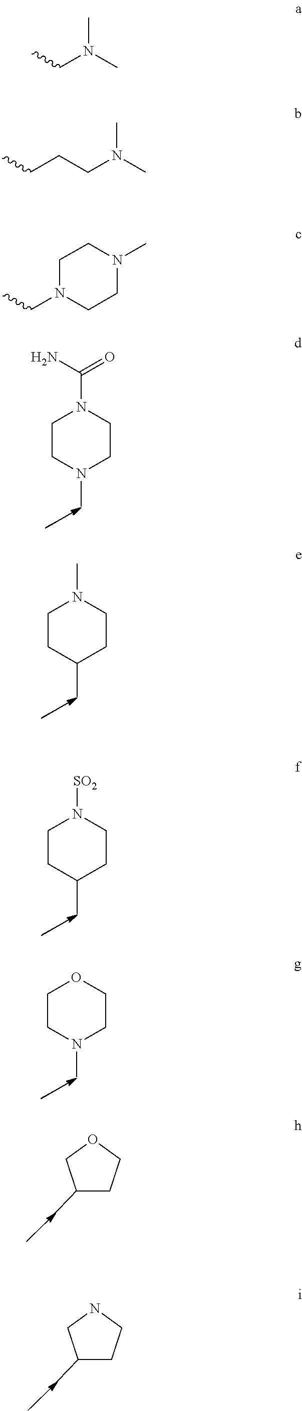

The term “cycloalkyl” means a saturated cyclic alkyl radical having from 3 to 10 carbon atoms. Representative cycloalkyls include cyclopropyl, 1-methylcyclopropyl, cyclobutyl, cyclopentyl, cyclohexyl, cycloheptyl, cyclooctyl, cyclononyl, and cyclodecyl. Cycloalkyl groups can be optionally substituted with one or more substituents.
The term “halogen” means —F, —Cl, —Br or —I.

### DETAILED DESCRIPTION

The present invention thus relates to a method for treating breast cancer in a subject in need thereof, wherein said method comprises administering to the subject a therapeutically effective amount of a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof.

In one embodiment, the present invention relates to a method for treating advanced breast cancer, preferably locally advanced or metastatic breast cancer, in a subject in need thereof, wherein said method comprises administering to the subject a therapeutically effective amount of a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof.

In another embodiment, the present invention relates to a method for treating triple-negative breast cancer in a subject in need thereof, wherein said method comprises administering to the subject a therapeutically effective amount of a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof.

In one embodiment, the present invention relates to a method for treating inflammatory breast cancer in a subject in need thereof, wherein said method comprises administering to the subject a therapeutically effective amount of a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof.

Tyrosine kinases are receptor type or non-receptor type proteins, which transfer the terminal phosphate of ATP to tyrosine residues of proteins thereby activating or inactivating signal transduction pathways. These proteins are known to be involved in many cellular mechanisms, which in case of disruption, lead to disorders such as abnormal cell proliferation and migration as well as inflammation. A tyrosine kinase inhibitor is a drug that inhibits tyrosine kinases, thereby interfering with signaling processes within cells. Blocking such processes can stop the cell growing and dividing.

In one embodiment, the tyrosine kinase inhibitor of the invention has the following formula (A):

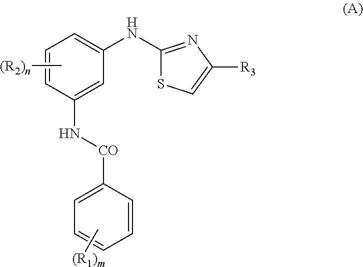

wherein
R₁ and R₂, are selected independently from hydrogen, halogen, a linear or branched alkyl, cycloalkyl group containing from 1 to 10 carbon atoms, trifluoromethyl, alkoxy, cyano, dialkylamino, and a solubilising group, m is 0-5 and n is 0-4;
the group R₃ is one of the following:
i. an aryl group such as phenyl or a substituted variant thereof bearing any combination, at any one ring position, of one or more substituents such as halogen, alkyl groups containing from 1 to 10 carbon atoms, trifluoromethyl, cyano and alkoxy;
ii. a heteroaryl group such as 2, 3, or 4-pyridyl group, which may additionally bear any combination of one or more substituents such as halogen, alkyl groups containing from 1 to 10 carbon atoms, trifluoromethyl and alkoxy;
iii. a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy;
   or a pharmaceutically acceptable salt or solvate thereof.

In one embodiment the tyrosine kinase inhibitor of the invention has general formula (B),

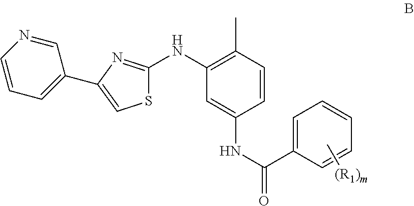

wherein:
R₁ is selected independently from hydrogen, halogen, a linear or branched alkyl, cycloalkyl group containing from 1 to 10 carbon atoms, trifluoromethyl, alkoxy, amino, alkylamino, dialkylamino, solubilising group;
m is 0-5;
   or a pharmaceutically acceptable salt or solvate thereof.

In one embodiment, the tyrosine kinase inhibitor of formula (B) is masitinib or a pharmaceutically acceptable salt or solvate thereof, more preferably masitinib mesilate.

The present invention thus also relates to masitinib or a pharmaceutically acceptable salt or solvate thereof, more preferably masitinib mesilate for treating breast cancer.

Pharmaceutically acceptable salts preferably are pharmaceutically acceptable acid addition salts, like for example with inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxy-benzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic, in particular methanesulfonic acid, or aromatic sulfonic acids, for example benzene-, p-toluene- or naphthalene-2-sulfonic acid.

Unless otherwise indicated, references to “mesilate” are used in the present invention to refer to a salt of methanesulfonic acid with a named pharmaceutical substance (such as compounds of formula (A) or (B)). Use of mesilate rather than mesylate is in compliance with the INNM (International nonproprietary names modified) issued by WHO (e.g. World Health Organization (February 2006). International Nonproprietary Names Modified. INN Working Document 05.167/3. WHO.). For example, masitinib mesilate means the methanesulfonic acid salt of masitinib.

Preferably, “masitinib mesilate” means the orally bioavailable mesilate salt of masitinib
CAS 1048007-93-7 (MsOH); C₂₈H₃₀N₆OS.CH₃SO₃H; MW 594.76:

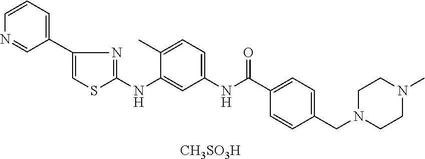

The chemical name for masitinib is 4-(4-methylpiperazin-1-ylmethyl)-N-[4-methyl-3-(4-pyridin-3ylthiazol-2-ylamino) phenyl]benzamide—CAS number 790299-79-5. Masitinib was described in U.S. Pat. No. 7,423,055 and EP1525200B1. A detailed procedure for the synthesis of masitinib mesilate is given in WO2008/098949.

Masitinib is a small molecule selectively inhibiting specific tyrosine kinases such as c-Kit, PDGFR, Lyn, Fyn and to a lesser extent the fibroblast growth factor receptor 3 (FGFR3), without inhibiting, at therapeutic doses, kinases associated with known toxicities (i.e. those tyrosine kinases or tyrosine kinase receptors attributed to possible tyrosine kinase inhibitor cardiac toxicity, including ABL, KDR and Src) (Dubreuil et al., 2009, PLoS ONE 2009.4(9):e7258). Moreover, masitinib is a c-Kit and platelet derived growth factor receptor (PDGFR) inhibitor with a potent anti mast cell action.

Masitinib's strong inhibitory effect on wild-type and juxtamembrane-mutated c-Kit receptors, results in cell cycle arrest and apoptosis of cell lines dependent on c-Kit signaling (Dubreuil et al., 2009, PLoS ONE, 4(9):e7258). Stem cell factor, the ligand of the c-Kit receptor, is a critical growth factor for mast cells; thus, masitinib is an effective antimastocyte, exerting a direct antiproliferative and pro-apoptotic action on mast cells through its inhibition of c-Kit signaling. Moreover, in vitro, masitinib demonstrated greater activity and selectivity against c-Kit than imatinib, inhibiting recombinant human wild-type c-Kit with an half inhibitory concentration (IC₅₀) of 200±40 nM and blocking stem cell factor-induced proliferation and c-Kit tyrosine phosphorylation with an IC₅₀ of 150±80 nM in Ba/F3 cells expressing human or mouse wild-type c-Kit.

Tyrosine kinase inhibitors of formula (A) or (B), in particular masitinib mesilate, can preferably be used as c-Kit inhibitors. The present invention thus also relates to a c-Kit inhibitor for treating breast cancer.

As used herein, the term “breast cancer” refers to all subtypes of invasive breast carcinomas, including triple negative breast cancer and inflammatory breast cancer.

In one embodiment, the method of the invention is for treating advanced breast cancer in a subject in need thereof, wherein the term “advanced breast cancer” encompasses locally advanced breast cancer and metastatic breast cancer. As used herein, the term “locally advanced breast cancer” refers to breast cancer that has spread locally to the area of the breast, such as, for example, to axillary lymph nodes, to lymph nodes near the breastbone, or to the skin of the breast, but has not spread to distant organs and tissues. In one embodiment, locally advanced breast cancers include stage III breast cancer (including stages IIIA, IIIB and IIIC breast cancer), and stages IIIB and IIIC inflammatory breast cancer.

In one embodiment, advanced breast cancer is stage III breast cancer, specifically stage IIIA, IIIB or IIIC breast cancer.

In another embodiment, advanced breast cancer is stage IV breast cancer (i.e. metastatic breast cancer).

In one embodiment, advanced breast cancer is stage III (including IIIB and IIIC) or stage IV inflammatory breast cancer.

In one embodiment, breast cancer is TNBC, wherein TNBC stands for triple-negative breast cancer. It is characterized by the absence of the estrogen-receptor (ER) and of the progesterone-receptor (PR), and by the absence of overexpression of the human epidermal growth factor receptor type 2 (HER2).

In one embodiment, breast cancer is locally advanced TNBC, such as, for example, stage III TNBC (including stages IIIA, IIIB and IIIC TNBC). In another embodiment, breast cancer is metastatic TNBC (i.e. stage IV TNBC).

In one embodiment, TNBC is inflammatory breast cancer.

In one embodiment, breast cancer is inflammatory breast cancer (IBC).

In one embodiment, breast cancer is advanced IBC. In one embodiment, breast cancer is locally advanced IBC. In another embodiment, breast cancer is metastatic IBC.

In one embodiment, inflammatory breast cancer is triple-negative inflammatory breast cancer.

In one embodiment, the subject was not treated previously with another treatment for breast cancer (i.e. the method of the invention is the first line treatment). In one embodiment, the subject was not treated previously with another systemic treatment for breast cancer (wherein a systemic treatment for breast cancer relates to treatment with a chemotherapeutic agent). In one embodiment, the subject was not previously treated for breast cancer by surgery or radiotherapy.

In another embodiment, the subject previously received one, two or more other treatments for breast cancer (i.e. the method of the invention is a second line, a third line or more). In one embodiment, the subject previously received one or more other treatments for breast cancer, but was unresponsive or did not respond adequately to these treatments, which means that there is no or too low therapeutic benefit induced by these treatments. Therapeutic benefits may include the fact of (1) slowing down or stopping the progression, aggravation, or deterioration of one or more symptoms of breast cancer; (2) bringing about ameliorations of the symptoms of breast cancer; (3) reducing the severity or incidence of breast cancer; or (4) curing breast cancer.

Examples of treatment for breast cancer include, but are not limited to, surgery (lumpectomy, partial or total mastectomy, modified radical mastectomy, lymph node surgery), tumor ablation, radiation therapy, chemotherapy (including treatment with the agents listed below), hormone therapy (including treatment with tamoxifen, estrogens or aromatase inhibitors), targeted therapy (including treatment with monoclonal antibodies (such as, for example, trastuzumab (especially in HER2 plus breast cancer), pertuzumab or ado-trastuzumab emtansine), other tyrosine kinase inhibitors than masitinib (such as, for example, lapatinib), and PARP inhibitors).

Examples of chemotherapeutic agents that may be used for treating breast cancer include, but are not limited to, abitrexate (Methotrexate), abraxane (Paclitaxel Albumin-stabilized Nanoparticle Formulation), ado-Trastuzumab Emtansine, adrucil (Fluorouracil), afinitor (Everolimus), anastrozole, aredia (Pamidronate Disodium), arimidex (Anastrozole), aromasin (Exemestane), carboplatin, capecitabine, cisplatin, Clafen (Cyclophosphamide), Cyclophosphamide, Cytoxan (Cyclophosphamide), Docetaxel, Doxorubicin Hydrochloride, Efudex (Fluorouracil), Ellence (Epirubicin Hydrochloride), Epirubicin Hydrochloride, Eribulin Mesylate, Everolimus, Exemestane, Fareston (Toremifene), Faslodex (Fulvestrant), Femara (Letrozole), Fluoroplex (Fluorouracil), Fluorouracil, Folex (Methotrexate), Folex PFS (Methotrexate), Fulvestrant, Gemcitabine Hydrochloride, Gemzar (Gemcitabine Hydrochloride), Goserelin Acetate, Halaven (Eribulin Mesylate), Herceptin (Trastuzumab), Ixabepilone, Ixempra (Ixabepilone), Kadcyla (Ado-Trastuzumab Emtansine), Lapatinib Ditosylate, Letrozole, Megace (Megestrol Acetate), Megestrol Acetate, Methotrexate, Methotrexate LPF (Methotrexate), Mexate (Methotrexate), Mexate-AQ (Methotrexate), mitoxantrone, Neosar (Cyclophosphamide), Nolvadex (Tamoxifen Citrate), Paclitaxel, Paclitaxel Albumin-stabilized Nanoparticle Formulation, Pamidronate Disodium, Perjeta (Pertuzumab), Pertuzumab, Tamoxifen Citrate, Taxol (Paclitaxel), Taxotere (Docetaxel), Trastuzumab, Toremifene, Tykerb (Lapatinib Ditosylate), Velban (Vinblastine Sulfate), Velsar (Vinblastine Sulfate), Vinblastine Sulfate, Xeloda (Capecitabine), Zoladex (Goserelin Acetate), Irinotecan, platinum-based chemotherapy, antimetabolites, anthracyclines and taxanes.

In one embodiment, the subject was previously treated with an anthracycline. Examples of anthracyclines include, but are not limited to, Daunorubicin, Doxorubicin, Epirubicin, Idarubicin, Valrubicin, and the anthracycline analog Mitoxantrone.

In one embodiment, the subject was previously treated with a taxane. Examples of taxanes include, but are not limited to, paclitaxel and docetaxel.

In one embodiment, the subject was previously treated with a platinum-based chemotherapeutic agent. Examples of platinum-based chemotherapeutic agents include, but are not limited to, cisplatin, carboplatin, oxaliplatin, satraplatin, picoplatin, nedaplatin, triplatin and lipoplatin.

In one embodiment, the subject was previously treated with an antimetabolite. Examples of antimetabolites include, but are not limited to, Azathioprine, Capécitabine, Cytarabine, Floxuridine, Fludarabine, Fluorouracile, Gemcitabine, Methotrexate, Pemetrexed.

According to a preferred embodiment, the method of treatment of the invention is a second line treatment, a third line treatment or more.

In one embodiment, the method of the invention further comprises administering a therapeutically effective amount of at least one chemotherapeutic agent. In one embodiment, the tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof and the at least one chemotherapeutic agent are administered simultaneously, separately or sequentially.

Indeed, the Applicant surprisingly demonstrated a synergetic effect of the combination of a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof such as, for example, masitinib mesilate with one or more chemotherapeutic agents. First, the Applicant showed in in vitro tests that, despite the absence of effect of the compound alone, the administration of a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof such as, for example, masitinib mesilate, sensitized cells to chemotherapeutic agents (see examples 1 to 3). Second, the Applicant showed in in vivo data that the administration to a subject of a combination of a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof such as, for example, masitinib mesilate with one or more chemotherapeutic agents allows increasing overall survival (see examples 4 to 7). In particular the Applicant showed in in vivo data that the administration to a subject suffering from inflammatory breast cancer of a combination of a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof such as, for example, masitinib mesilate with one or more chemotherapeutic agents allows increasing overall survival (see example 4).

The present invention thus also relates to a method for treating breast cancer in a subject in need thereof, comprising administering to the subject a therapeutically effective amount of tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof, or a pharmaceutically acceptable salt or solvate thereof in combination with a therapeutically effective amount of at least one chemotherapeutic agent.

In one embodiment, the present invention relates to a method for treating inflammatory breast cancer (IBC) in a subject in need thereof, comprising administering to the subject a therapeutically effective amount of tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof, or a pharmaceutically acceptable salt or solvate thereof in combination with a therapeutically effective amount of at least one chemotherapeutic agent.

Examples of chemotherapeutic agents that may be used in combination with the tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof of the invention are listed hereinabove.

In one embodiment, the chemotherapeutic agent is selected from the group comprising antimetabolites (preferably capecitabine and/or gemcitabine), anthracyclines, taxanes, platinum-based chemotherapeutic agents (preferably cisplatin and/or carboplatin) and mixtures thereof.

In one embodiment, the chemotherapeutic agent is selected from the group comprising gemcitabine, doxorubicin, cisplatin, carboplatin, capecitabine, irinotecan, mitoxantrone and mixtures thereof.

In one embodiment, the chemotherapeutic agent is selected from anthracyclines. In one embodiment, the chemotherapeutic agent is selected from the group consisting of doxorubicin, epirubicin, pixantrone, losoxantrone, mitoxantrone and daunorubicin, or any mixtures thereof. In another embodiment, the chemotherapeutic agent is doxorubicin or mitoxantrone.

In one embodiment, breast cancer is TNBC, and the chemotherapeutic agent is selected from antimetabolites (preferably capecitabine and/or gemcitabine), anthracyclines (preferably doxorubicin and/or mitoxantrone), taxanes, platinum-based chemotherapeutic agents (preferably cisplatin and/or carboplatin) and mixtures thereof. In one embodiment, breast cancer is TNBC, and the chemotherapeutic agent is selected from cisplatin, gemcitabine, carboplatin, doxorubicin, mitoxantrone and mixtures thereof, such as, for example, a mixture of gemcitabine and carboplatin.

The present invention thus also relates to a method for treating TNBC in a subject in need thereof, comprising administering to the subject:
a therapeutically effective amount of tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof, or a pharmaceutically acceptable salt or solvate thereof, preferably masitinib mesilate;
in combination with
a therapeutically effective amount of at least one chemotherapeutic agent selected from antimetabolites, anthracyclines, taxanes, platinum-based chemotherapeutic agents and mixtures thereof, preferably cisplatin, gemcitabine, carboplatin, doxorubicin, mitoxantrone and mixtures thereof, such as, for example, a mixture of gemcitabine and carboplatin.

In one embodiment, the method for treating TNBC of the invention comprises administering masitinib mesilate and cisplatin. In another embodiment, the method for treating TNBC of the invention comprises administering masitinib mesilate and gemcitabine. In another embodiment, the method for treating TNBC of the invention comprises administering masitinib mesilate and carboplatin. In another embodiment, the method for treating TNBC of the invention comprises administering masitinib mesilate, gemcitabine and carboplatin. In another embodiment, the method for treating TNBC of the invention comprises administering masitinib mesilate and anthracyclines (preferably doxorubicin or mitoxantrone).

In one embodiment, breast cancer is metastatic breast cancer, and the chemotherapeutic agent is selected from is selected from antimetabolites (preferably capecitabine and/or gemcitabine), anthracyclines (preferably doxorubicin and/or mitoxantrone), taxanes, platinum-based chemotherapeutic agents (preferably cisplatin and/or carboplatin) and mixtures thereof. In one embodiment, breast cancer is metastatic breast cancer, and the chemotherapeutic agent is selected from gemcitabine, capecitabine, carboplatin doxorubicin, mitoxantrone and mixtures thereof.

The present invention thus also relates to a method for treating metastatic breast cancer in a subject in need thereof, comprising administering to the subject:
a therapeutically effective amount of tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof or a pharmaceutically acceptable salt or solvate thereof, preferably masitinib mesilate;
in combination with
a therapeutically effective amount of at least one chemotherapeutic agent selected from antimetabolites, anthracyclines, taxanes, platinum-based chemotherapeutic agents and mixtures thereof, preferably gemcitabine, capecitabine, carboplatin, doxorubicin, mitoxantrone and mixtures thereof.

In one embodiment, the method for treating metastatic breast cancer of the invention comprises administering masitinib mesilate and anthracyclines (preferably doxorubicin or mitoxantrone). In another embodiment, the method for treating metastatic breast cancer of the invention comprises administering masitinib mesilate and capecitabine. In another embodiment, the method for treating metastatic breast cancer of the invention comprises administering masitinib mesilate and gemcitabine. In another embodiment, the method for treating metastatic breast cancer of the invention comprises administering masitinib mesilate and carboplatin.

In one embodiment, breast cancer is inflammatory breast cancer, and the chemotherapeutic agent is selected from antimetabolites (preferably capecitabine and/or gemcitabine), anthracyclines (preferably doxorubicin and/or mitoxantrone), taxanes, platinum-based chemotherapeutic agents (preferably cisplatin and/or carboplatin) and mixtures thereof. In one embodiment, breast cancer is inflammatory breast cancer, and the chemotherapeutic agent is selected from cisplatin, gemcitabine, capecitabine, carboplatin, doxorubicin, mitoxantrone and mixtures thereof.

The present invention thus also relates to a method for treating inflammatory breast cancer in a subject in need thereof, comprising administering to the subject:
a therapeutically effective amount of tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof or a pharmaceutically acceptable salt or solvate thereof, preferably masitinib mesilate;
in combination with
a therapeutically effective amount of at least one chemotherapeutic agent selected from antimetabolites, anthracyclines, taxanes, platinum-based chemotherapeutic agents, anthracyclines and mixtures thereof, preferably cisplatin, gemcitabine, capecitabine, carboplatin, doxorubicin, mitoxantrone and mixtures thereof.

In one embodiment, the method for treating inflammatory breast cancer of the invention comprises administering masitinib mesilate and capecitabine. In another embodiment, the method for treating inflammatory breast cancer of the invention comprises administering masitinib mesilate and gemcitabine. In another embodiment, the method for treating inflammatory breast cancer of the invention comprises administering masitinib mesilate and cisplatin. In another embodiment, the method for treating inflammatory breast cancer of the invention comprises administering masitinib mesilate and carboplatin. In another embodiment, the method for treating inflammatory breast cancer of the invention comprises administering masitinib mesilate and anthracyclines (preferably doxorubicin or mitoxantrone).

In one embodiment, the therapeutically effective amount of a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof, or a pharmaceutically acceptable salt or solvate thereof ranges from about 1 to about 20 mg/kg/day, preferably from about 3 to about 12 mg/kg/day, and more preferably from about 4.5 to about 9 mg/kg/day. In one embodiment, the therapeutically effective amount of a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof or a pharmaceutically acceptable salt or solvate thereof ranges from about 6 to about 9 mg/kg/day. In one embodiment, the therapeutically effective amount of a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof or a pharmaceutically acceptable salt or solvate thereof is of about 4.5 mg/kg/day or of about 6 mg/kg/day or of about 7.5 mg/kg/day or of about 9 mg/kg/day.

Unless otherwise indicated, any dose indicated herein refers to the amount of active ingredient as such, not to its salt form. Therefore, given that the tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof, dose in mg/kg/day used in the described dose regimens refers to the amount of active ingredient tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof, compositional variations of a pharmaceutically acceptable salt or solvate of tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof will not change the said dose regimens.

In one embodiment, the tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof or a pharmaceutically acceptable salt or solvate thereof is orally administered.

In one embodiment, the tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof or a pharmaceutically acceptable salt or solvate thereof is administered once or twice a day.

In one embodiment, the therapeutically effective amount of a chemotherapeutic agent ranges from about 10 to 5000 mg/m², preferably from about 25 to about 1500 mg/m², and more preferably from about 75 to about 1250 mg/m².

In one embodiment, the therapeutically effective amount of cisplatin ranges from about 1 to 300 mg/m², preferably from about 25 to about 150 mg/m², and more preferably about 75 mg/m².

In one embodiment, the therapeutically effective amount of gemcitabine ranges from about 100 to 2000 mg/m², preferably from about 500 to about 1500 mg/m², and more preferably from about 750 to about 1250 mg/m². In one embodiment, the therapeutically effective amount of gemcitabine is of about 750 mg/m², of about 1000 mg/m² or of about 1250 mg/m².

In one embodiment, the therapeutically effective amount of carboplatin ranges from about AUC 1 to about AUC 10, preferably from about AUC 3 to about AUC 7, and more preferably about AUC 5. As used herein, the term “AUC” is for “area under curve” dose, and may be easily determined by the skilled artisan.

In one embodiment, the therapeutically effective amount of capecitabine ranges from about 200 to 2000 mg/m², preferably from about 750 to about 1500 mg/m², and more preferably about 1250 mg/m².

In one embodiment, the therapeutically effective amount of mitoxantrone ranges from about 3 to 100 mg/m², preferably from about 10 to about 25 mg/m², and more preferably about 12 mg/m².

In one embodiment, the therapeutically effective amount of doxorubicin ranges from about 10 to 150 mg/m², preferably from about 40 to about 75 mg/m², and more preferably about 60 mg/m².

In one embodiment, the chemotherapeutic agent is injected, such as, for example, by intravenous injection or infusion.

In one embodiment, the chemotherapeutic agent is orally administered. In one embodiment, the chemotherapeutic agent is capecitabine, and is orally administered.

In one embodiment, the chemotherapeutic agent is administered once or twice a day, or 1, 2, 3, 4, 5, 6, 7 times per week, or 1, 2, 3, 4, 5, 6, 7 times per two weeks or 1, 2, 3, 4, 5, 6, 7 times per months. In one embodiment, the administration schedule may include days or weeks periods wherein the chemotherapeutic agent is not administered. For example, the chemotherapeutic agent may be administered at first day of each week, or at first day of week 1 and week 2 of a 3 weeks cycle, or every days of a week followed by a 7-day rest period, and the like. The skilled artisan may easily adapt the administration schedule, according, for example, to previous treatment history of the patient, to the severity of the disease to be treated, to the nature of the chemotherapeutic agent and the like.

Another object of the invention is a composition comprising a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof or a pharmaceutically acceptable salt or solvate thereof and at least one chemotherapeutic agent. In one embodiment, the tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof is masitinib, preferably masitinib mesilate. In one embodiment, the chemotherapeutic agent is selected from antimetabolites (preferably capecitabine and/or gemcitabine), anthracyclines (preferably doxorubicin and/or mitoxantrone), taxanes, platinum-based chemotherapeutic agents (preferably cisplatin and/or carboplatin) and mixtures thereof. In another embodiment, the chemotherapeutic agent is selected from the group comprising gemcitabine, doxorubicin, cisplatin, carboplatin, capecitabine, irinotecan, mitoxantrone and mixtures thereof.

Another object of the invention is a pharmaceutical composition comprising a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof or a pharmaceutically acceptable salt or solvate thereof and at least one chemotherapeutic agent, in combination with at least one pharmaceutically acceptable carrier. In one embodiment, the tyrosine kinase inhibitor is masitinib, preferably masitinib mesilate. In one embodiment, the chemotherapeutic agent is selected from antimetabolites (preferably capecitabine and/or gemcitabine), anthracyclines (preferably doxorubicin and/or mitoxantrone), taxanes, platinum-based chemotherapeutic agents (preferably cisplatin and/or carboplatin) and mixtures thereof. In another embodiment, the chemotherapeutic agent is selected from the group comprising gemcitabine, doxorubicin, cisplatin, carboplatin, capecitabine, irinotecan, mitoxantrone and mixtures thereof.

Another object of the invention is a medicament comprising a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof or a pharmaceutically acceptable salt or solvate thereof and at least one chemotherapeutic agent. In one embodiment, the tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof is masitinib, preferably masitinib mesilate. In one embodiment, the chemotherapeutic agent is selected from antimetabolites (preferably capecitabine and/or gemcitabine), anthracyclines (preferably doxorubicin and/or mitoxantrone), taxanes, platinum-based chemotherapeutic agents (preferably cisplatin and/or carboplatin) and mixtures thereof. In another embodiment, the chemotherapeutic agent is selected from the group comprising gemcitabine, doxorubicin, cisplatin, carboplatin, capecitabine, irinotecan, mitoxantrone and mixtures thereof.

Another object of the invention is a kit of part comprising two parts, wherein the first part comprises a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof or a pharmaceutically acceptable salt or solvate thereof and wherein the second part comprises at least one chemotherapeutic agent. In one embodiment, the tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof is masitinib, preferably masitinib mesilate. In one embodiment, the chemotherapeutic agent is selected from antimetabolites (preferably capecitabine and/or gemcitabine), anthracyclines (preferably doxorubicin and/or mitoxantrone), taxanes, platinum-based chemotherapeutic agents (preferably cisplatin and/or carboplatin) and mixtures thereof. In another embodiment, the chemotherapeutic agent is selected from the group comprising gemcitabine, doxorubicin, cisplatin, carboplatin, capecitabine, irinotecan, mitoxantrone and mixtures thereof.

In one embodiment, the composition, pharmaceutical composition, medicament or kit of part of the invention comprises or consists in masitinib mesilate and cisplatin. In another embodiment, the composition, pharmaceutical composition, medicament or kit of part of the invention comprises or consists in masitinib mesilate and gemcitabine. In another embodiment, the composition, pharmaceutical composition, medicament or kit of part of the invention comprises or consists in masitinib mesilate and carboplatin. In another embodiment, the composition, pharmaceutical composition, medicament or kit of part of the invention comprises or consists in masitinib mesilate, gemcitabine and carboplatin. In another embodiment, the composition, pharmaceutical composition, medicament or kit of part of the invention comprises or consists in masitinib mesilate and capecitabine. In another embodiment, the composition, pharmaceutical composition, medicament or kit of part of the invention comprises or consists in masitinib mesilate and doxorubicin. In another embodiment, the composition, pharmaceutical composition, medicament or kit of part of the invention comprises or consists in masitinib mesilate and mitoxantrone.

In one embodiment of the invention, the composition, pharmaceutical composition, medicament or kit of part of the invention comprises an amount of a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof ranging from about 10 to about 500 mg, preferably from about 50 to about 300 mg, and more preferably from about 100 to about 200 mg.

In one embodiment of the invention, the composition, pharmaceutical composition, medicament or kit of part of the invention comprises an amount of masitinib ranging from about 10 to about 500 mg, preferably from about 50 to about 300 mg, and more preferably from about 100 to about 200 mg. In one embodiment, the composition, pharmaceutical composition, medicament or kit of part of the invention comprises an amount of masitinib of about 100 mg (corresponding to an amount of masitinib mesilate of about 119.3 mg). In another embodiment, the composition, pharmaceutical composition, medicament or kit of part of the invention comprises an amount of masitinib of about 200 mg (corresponding to an amount of masitinib mesilate of about 238.5 mg).

In one embodiment, the composition, pharmaceutical composition, medicament of the invention or the first and/or second part of the kit of part of the invention is in a form adapted for oral administration.

Examples of forms adapted for oral administration include, but are not limited to, tablets, orodispersing/orodispersing tablets, effervescent tablets, powders, granules, pills (including sugarcoated pills), dragees, capsules (including soft gelatin capsules), syrups, liquids, gels or other drinkable solutions, suspensions, slurries, liposomal forms and the like.

In one embodiment, the composition, pharmaceutical composition, medicament of the invention or the first and/or second part of the kit of part of the invention is in a form adapted for injection, such as, for example, for intramuscular, subcutaneous, intradermal, transdermal or intravenous injection or infusion.

Examples of forms adapted for injection include, but are not limited to, solutions, such as, for example, sterile aqueous solutions, dispersions, emulsions, suspensions, solid forms suitable for using to prepare solutions or suspensions upon the addition of a liquid prior to use, such as, for example, powder, liposomal forms and the like.

In one embodiment, the part of the kit of part comprising the tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof or a pharmaceutically acceptable salt or solvate thereof is in a form adapted for oral administration, while the second part of the kit of part (comprising at least one chemotherapeutic agent) is in a form adapted for injection.

In another embodiment, the part of the kit of part comprising the tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof or a pharmaceutically acceptable salt or solvate thereof and the second part of the kit of part (comprising at least one chemotherapeutic agent) are both in a form adapted for oral administration.

The present invention further relates to a composition, a pharmaceutical composition, a medicament or a kit of part as described hereinabove for treating breast cancer, or for use in treating breast cancer.

In one embodiment the present invention further relates to a composition, a pharmaceutical composition, a medicament or a kit of part as described hereinabove for treating inflammatory breast cancer, or for use in treating inflammatory breast cancer.

In one embodiment of the invention, the composition, pharmaceutical composition, medicament or kit of part as described hereinabove is for use in the method for treating breast cancer of the invention.

In one embodiment of the invention, the composition, pharmaceutical composition, medicament or kit of part as described hereinabove is for use in the method for treating inflammatory breast cancer of the invention.

Masitinib is a small molecule drug, selectively inhibiting specific tyrosine kinases such as c-Kit, platelet-derived growth factor receptor (PDGFR), LYN, and FYN, without inhibiting, at therapeutic doses, kinases associated with known toxicities (i.e. those tyrosine kinases or tyrosine kinase receptors attributed to possible tyrosine kinase inhibitor cardiac toxicity, including ABL, KDR and Src) (Dubreuil et al., 2009, PLoS ONE 2009.4(9):e7258).

In one embodiment, the method of the invention comprises inhibiting tyrosine kinases, preferably selected from the group consisting of c-Kit, LYN, FYN and PDGFR α and β, thereby treating breast cancer.

The present invention thus also relates to a method for inhibiting tyrosine kinases, preferably selected from the group consisting of c-Kit, LYN, FYN and PDGFR α and β in a breast cancer patient, thereby treating breast cancer, wherein said method comprises administering a therapeutically effective amount of masitinib or a pharmaceutically acceptable salt or solvate thereof.

In one embodiment, the method of the invention comprises inhibiting c-Kit. In one embodiment, the method of the invention comprises inhibiting LYN. In one embodiment, the method of the invention comprises inhibiting FYN. In one embodiment, the method of the invention comprises inhibiting PDGFR α and β, in particular inhibiting the in vitro protein kinase activity of PDGFR-α and β.

Masitinib's main kinase target is c-Kit, for which it has been shown to exert a strong inhibitory effect on wild-type and juxtamembrane-mutated c-Kit receptors, resulting in cell cycle arrest and apoptosis of cell lines dependent on c-Kit signaling (Dubreuil et al., 2009, PLoS ONE, 4(9):e7258). In vitro, masitinib demonstrated high activity and selectivity against c-Kit, inhibiting recombinant human wild-type c-Kit with an half inhibitory concentration (IC₅₀) of 200±40 nM and blocking stem cell factor-induced proliferation and c-Kit tyrosine phosphorylation with an IC₅₀ of 150±80 nM in Ba/F3 cells expressing human or mouse wild-type c-Kit. In addition to its anti-proliferative properties, masitinib can also regulate the activation of mast cells through its targeting of Lyn and Fyn, key components of the transduction pathway leading to IgE induced degranulation (Gilfillan et al., 2006, Nat Rev Immunol, 6:218-230; Gilfillan et al., 2009, Immunological Reviews, 228:149-169). This can be observed in the inhibition of FcεRI-mediated degranulation of human cord blood mast cells (Dubreuil et al., 2009, PLoS ONE; 4(9):e7258). Masitinib is also an inhibitor of PDGFR α and β receptors. Recombinant assays show that masitinib inhibits the in vitro protein kinase activity of PDGFR-α and β with IC₅₀ values of 540±60 nM and 800±120 nM. In Ba/F3 cells expressing PDGFR-α, masitinib inhibited PDGF-BB-stimulated proliferation and PDGFR-α tyrosine phosphorylation with an IC₅₀ of 300±5 nM.

In oncology indications for which masitinib's tyrosine kinase targets are not the main oncogenic drivers the main mode of action of masitinib is through modulation of the immune response. Experimental data indicate that masitinib is capable of modulating the immune response in such a way as to positively impact on physiological disturbances such as oxidative stress (Adenis A, et al. Ann Oncol. 2014 September; 25(9):1762-9). In particular, masitinib induces an anti-tumoral Th1 immune response via recruitment of macrophages with a potential anti-tumoral activity within the tumor and also modulates the tumor microenvironment through its inhibition of mast cell activity with reduced release of M2-polarizing cytokines (pro-tumoral), as well as other factors favoring metastasis and angiogenesis. Subsequent anti-tumoral activity within the tumor and tumor microenvironment confers conditions conducive to retarding aggressiveness and dissemination of the tumor in a manner independent of association with any particular active chemotherapeutic agent.

More specifically, recent experimental data have demonstrated that masitinib also acts by modulating immune response. In particular, masitinib induces an anti-tumoral Th1 immune response, due to the following mechanisms of action: (i) masitinib acts on macrophage, by increasing both the release of chemoattractants which attracts macrophages to the tumor site (such as, for example, CCL2), and the expression of M1-polarizing cytokines, such as, for example, CXCL9 and CXCL10; (ii) masitinib inhibits mast cell proliferation and degranulation and thereby reduces the release of M2-polarizing cytokines, as well as other factors favoring metastasis and angiogenesis (such as VEGF); and (iii) masitinib increases cytotoxic NK activity and IFN gamma release through its interaction with dendritic cells.

In one embodiment, the method of the invention comprises inducing an anti-tumoral Th1 immune response, thereby treating breast cancer.

The present invention thus also relates to a method for inducing an anti-tumoral Th1 immune response in a breast cancer patient, thereby treating breast cancer, wherein said method comprises administering a therapeutically effective amount of masitinib or a pharmaceutically acceptable salt or solvate thereof.

In one embodiment, the method of the invention comprises increasing the release of chemoattractants which attracts macrophages to the tumor site (such as, for example, CCL2), and/or increasing the expression of M1-polarizing cytokines, such as, for example, CXCL9 and CXCL10.

In one embodiment, the method of the invention comprises inhibiting mast cell proliferation and degranulation and thereby reducing the release of M2-polarizing cytokines, as well as other factors favoring metastasis and angiogenesis (such as VEGF).

In one embodiment, the method of the invention comprises increasing cytotoxic NK activity and IFN gamma release through the interaction of masitinib with dendritic cells.

In one embodiment, the method of the invention comprises (i) inhibiting tyrosine kinases, preferably selected from the group consisting of c-Kit, LYN, FYN and PDGFR α and β and (ii) inducing an anti-tumoral Th1 immune response, thereby treating breast cancer.

The present invention thus also relates to a method for (i) inhibiting tyrosine kinases, preferably selected from the group consisting of c-Kit, LYN, FYN and PDGFR α and β and (ii) inducing an anti-tumoral Th1 immune response, in a breast cancer patient, thereby treating breast cancer, wherein said method comprises administering a therapeutically effective amount of masitinib or a pharmaceutically acceptable salt or solvate thereof.

In one embodiment, the method of the invention comprises inhibiting c-Kit signaling pathways. In one embodiment, the method of the invention comprises inhibiting LYN signaling pathways. In one embodiment, the method of the invention comprises inhibiting FYN signaling pathways. In one embodiment, the method of the invention comprises inhibiting PDGF signaling pathways. As used herein, the terms “signaling pathway” refers to a group of molecules in a cell that work together to control one or more cell functions (such as, for example cell division or cell death). After the first molecule in a pathway receives a signal, it activates another molecule. This process is repeated until the last molecule is activated and the cell function is carried out.

### BRIEF DESCRIPTION OF THE DRAWINGS

FIG. 1 is a histogram showing the sensitivity of resistant Mia Paca-2 cells to masitinib, imatinib and dasatinib.

FIG. 2 is a histogram showing the sensitivity of Mia Paca-2 cells to the combination treatment of gemcitabine plus masitinib, imatinib or dasatinib.

### EXAMPLES

The present invention is further illustrated by the following examples.

### Example 1: Masitinib Sensitized Breast Cancer Cell Lines to Chemotherapies

### Materials and Methods

### Compounds

Masitinib mesilate (having the molecular formula C₂₈H₃₀N₆OS.CH₄O₃S) presents as a white powder. Stock solution of 20 mM in DMSO was stored at −80° C. Gemcitabine (2′,2′,-dofluoro-2′,-deoxycytidine) was from Eli Lilly and is a nucleoside analogue of deoxycytidine that interferes with DNA synthesis. The other agents were purchased from Sigma Aldrich Corporation: anti-topoisomerase I (Irinotecan), alkylant agents such as platinum salt (Carboplatin), and doxorubicin (anthracycline antibiotic). These agents are commonly used as treatment for various tumor types either as single agent or in combination regimen.

### Cell Culture

Breast cancer cell lines (generous gift from Dr. Patrice Dubreuil, UMR 599 INSERM, Marseille, France) were cultured as monolayers in RPMI 1640 medium, containing L-glutamine supplemented with 100 U/mL penicillin and 100 μg/mL streptomycin, and 10% v/v heat-inactivated fetal calf serum (AbCys Lot S02823S1800) under standard culture conditions (5% CO2, 95% air in humidified chamber at 37° C.). During proliferation assay, all cells were grown in medium containing 1% FCS.

### Experimental Design

Colorimetric cell proliferation and viability assay (reagent WST-1 purchased from Roche cat N^{o} 1644807)—Cells were washed once and resuspended in RPMI 1% FCS. Cells were plated at 1×10⁴/50 μl per well of a 96 well plate. Drug dilutions were prepared in a 96 well plate and obtained by sequential dilutions of masitinib or gemcitabine in RPMI 1. Treatment was started by the addition of 50 μl of a 2× concentrated drug solution to a final volume of 100 μl. For treatment with combination of masitinib mesilate and cytotoxic agents, the cells were first resuspended in medium RPMI 1% FCS containing masitinib at the concentrations of 0, 2, 5 and 10 μM. 1×10⁴ cells/50 μl were plated per well of a 96 wells plate and the plates placed in the incubator o/n before treatment with cytotoxic agents. Cytotoxic agent treatment was initiated by addition of 50 μl of a 2× drug dilution (and containing the respective masitinib drug concentration) to a final volume of 100 μl. Masitinib final concentrations remained 0, 2, 5 and 10 μM. After incubating for 72 hours at 37° C., 10 μl of a ½ dilution of WST-1 reagent was added to each well and the plates were returned to the incubator for an additional 4 hours. The absorbance of the samples was measured at 490 nm using an EL800 Universal microplate reader (Bio-Tek Instruments Inc.). A background control without cells was used as a blank. The positive control of the assay corresponds to the cell proliferation obtained in the absence of drug treatment (100% proliferation). Each sample was done in duplicate, the absorbance values were transferred to an excel file, the average and standard deviation of the duplicates were calculated and expressed as a percentage of the proliferation obtained in absence of treatment. The results presented are representative of a minimum of four experiments. The sensitization factor/Index is calculated by dividing the IC₅₀ of the chemotherapeutic agent alone by the IC₅₀ of the chemotherapeutic agent used in combination with masitinib mesilate.

### Results

In order to assess the benefits of using masitinib mesilate in combination therapy for cancer treatment, preclinical studies involving tumor cell lines were performed. The project consisted to evaluate the ability of masitinib mesilate to sensitize breast cancer cell lines to cytotoxic agents using in vitro proliferation assays.

We used a large panel of cytotoxic agents that exert their cytotoxicity through different mechanisms. These agents included the conventional chemotherapies gemcitabine (GCB) and doxorubicin (DOX) as well as non-standard chemotherapeutic agents such as irinotecan (CPT-11) and platinum salts carboplatin (CPT).

### Masitinib Mesilate is not Active as Single Agent

Breast cancer cell lines were first analyzed for their sensitivity to masitinib mesilate when used as single agent. This analysis showed that breast cancer cell lines were not sensitive to masitinib mesilate (IC₅₀>6 μM) suggesting that proliferation/survival of the cell lines examined may not be dependent on the expression masitinib mesilate main targets PDGFRα/β and c-Kit. Based on these data, masitinib mesilate was used at concentrations of 5 and 10 μM in the following combinatory experiments.

### Masitinib Mesilate Sensitizes Breast Cancer Cells to Gemcitabine

To determine the IC₅₀ of gemcitabine as single agent or in association with masitinib mesilate, breast cancer cell lines grown in 1% FCS were pre-treated with solvent control (DMSO) or masitinib mesilate for about 12-16 hours before being exposed to different doses of the chemotherapeutic agent.

Results are shown in Table 1.

TABLE 1
Masitinib mesilate sensitizes breast
tumor cell lines to gemcitabine (GCB)

| IC₅₀ μM | GCB | GCB plus | |
|---|---|---|---|
| Cell lines | μM | Masitinib mesilate | SI |
| BT20 | 50 | 5-10 | 5-10 |
| BT474 | 100 | 10-50 | 2-10 |
| MDAMB134 | 100 | 10-50 | 2-10 |
| MDAMB231 | 100 | 50 | 2 |

SI = Sensitization Index

A good masitinib mesilate sensitization to gemcitabine is observed in these cell lines resistant to gemcitabine. Moreover, the addition of masitinib mesilate lowers the IC₅₀ of gemcitabine to clinically achievable concentrations.

### Masitinib Mesilate Sensitizes Breast Cancer Cells to Doxorubicin

The ability of masitinib mesilate to sensitize breast cancer cell lines to the action of the anthracycline doxorubicin (Adriamycin) was next assessed. Summary of the results is presented in Table 2.

TABLE 2
Masitinib mesilate sensitizes breast
tumor cell lines to doxorubicin (DOX)

| IC₅₀ (μM) | DOX | DOX plus | |
|---|---|---|---|
| Cell lines | μM | Masitinib mesilate | SI |
| MDAMB231 | 1 | 0.1 | 10 |
| MDAMB134 | 1 | 0.1-0.5 | 2-10 |
| HCC1937 | 1 | 0.1-0.5 | 2-10 |
| MCF-7 | 1 | 0.25-0.1 | 4-10 |

SI = Sensitization Index

Interestingly these four cell lines appear to be resistant to doxorubicin and a good sensitization is observed when masitinib mesilate was added. The presence of masitinib mesilate lowers the IC₅₀ of doxorubicin to clinically achievable concentrations.

### Masitinib Mesilate Sensitizes Breast Cancer Cell Lines to Carboplatin

We next tested the ability of masitinib mesilate to sensitize breast cancer cell lines to the action of carboplatin. Summary of the results is presented in Table 3.

TABLE 3
Masitinib mesilate sensitizes breast
tumor cell lines to carboplatin (CPT)

| IC₅₀ (μM) | CPT | CPT plus | |
|---|---|---|---|
| Cell lines | μM | Masitinib mesilate | SI |
| MDAMB134 | >100 | 10 | >10 |
| MDAMB231 | >100 | 30 | >3 |
| BT474 | >100 | 50 | >2 |
| BrCa-MZ-01 | 100 | 5 | 20 |
| BT20 | 100 | 20 | 5 |

SI = Sensitization Index

The cell lines MDAMB134 and 231, BT474, BrCa-MZ-01 and BT20 are resistant to carboplatin and the addition of masitinib mesilate significantly enhances the sensitivity of these cell lines to carboplatin.

### Masitinib Mesilate Sensitizes Breast Cancer Cells to Irinotecan

We next assessed the ability of masitinib mesilate to sensitize breast cancer cell lines to the action of an inhibitor of topoisomerase I irinotecan. Summary of the results is presented in Table 4.

TABLE 4
Masitinib mesilate sensitizes breast tumor
cell lines to irinotecan (CPT-11)

| IC₅₀ (μM) | CPT-11 | CPT-11 plus | |
|---|---|---|---|
| Cell lines | μM | Masitinib mesilate | SI |
| MCF-7 | 100 | 5-20 | 5-20 |
| MDAMB134 | 30 | 10 | 3 |
| MDAMB231 | 30 | 10 | 3 |

SI = Sensitization Index

Masitinib mesilate efficiently sensitizes MCF-7 cell line to irinotecan while sensitizing to a lesser extent MDAMB134 and 231 cell lines.

These results thus demonstrate that, surprisingly, masitinib mesilate is able to sensitize breast cancer cell lines to cytotoxic agents in vitro, despite its absence of activity when used alone. Therefore, these results highlight the synergistic effect of the combination of masitinib mesilate and cytotoxic agents.

### Example 2: Masitinib Sensitized Breast Cancer Cell Lines to Chemotherapies

Mitoxantrone is an anthracycline analog and type II topoisomerase inhibitor. Mitoxantrone is an intravenous treatment for advanced-stage breast cancer. Other drugs of this class include doxorubicin, epirubicin, pixantrone, losoxantrone, daunorubicin and other anthracyclines.

### Materials and Methods

Two canine mammary tumor (CMT) cell lines were evaluated, CMT-U27 and CMT-U309. CMT-U27 is a highly metastatic canine mammary carcinoma cell line while CMT-U309 is a non-metastatic spindle-cell cell line. Cell lines were maintained in Dulbecco's modified Eagles Medium-F12 supplemented with 10% fetal bovine serum and antimicrobials (penicillin [100 U/mL] and streptomycin [0.1 mg/mL]). Cell lines were maintained in a humidified atmosphere of 5% carbon dioxide at 37° C.

Masitinib mesilate was dissolved in 100% DMSO at a concentration of 20 mM to create a stock solution, which was then protected from light and kept at −20° C. The stock solution was further diluted with culture media prior to use in tissue culture, DMSO concentration did not exceed 0.2% volume per condition. Mitoxantrone dihydrochloride was dissolved in Dulbecco's modified Eagles Medium-F12 at a concentration of 3.2 mM to create a stock solution. Solutions were prepared newly prepared from the stock solution for each experiment.

Cells were seeded at a density of 1.0×10⁴ cells/well (final volume, 100 μL/well) in three replicates. After 24 hours, 100 μL of medium containing (DMSO) (control wells) or various concentrations of masitinib and mitoxantrone were added to each well to create final masitinib and mitoxantrone concentrations of 0.25, 0.5, 1, 2, 4, 8, 16 and 32 μM, and the cells were incubated for an additional 24, 48 and 72 hours. At the end of treatment, 10 μl of MTT solution (5 mg/ml in PBS) was added to each well. The plates were incubated for 4 hours in a humidified atmosphere at 37° C. with 5% CO₂. After that incubation, cell proliferation was assessed by use of a commercial cell proliferation MTT [3-(4,5-dimethylthiazol-2-yl)-2,5-diphenyltrazolium bromide] kit. All samples were assayed in triplicate, and the mean for each experiment was calculated. The effect of each compound on growth inhibition was assessed as percent cell viability where vehicle-treated cells were taken as 100% viable. Cell proliferation was expressed as a percentage of the control wells by use of the following equation: (absorbance of sample wells/absorbance of control wells)×100. The mean of triplicate experiments for each dose was used to calculate IC₅₀ as determined by CalcuSyn software. Proliferation assays for the combined incubation of masitinib with mitoxantrone used the same procedure except that the cells were incubated for 72 hours.

The multiple drug effect analysis of Chou and Talalay, which is based on the median-effect principle, was used to examine the nature of the interaction observed between masitinib and mitoxantrone (Chou T C, Talalay P. Adv Enzyme Regul. 1984; 22:27-55) (Chou T C. Cancer Res. 2010 Jan. 15; 70(2):440-6). Determination of the synergistic versus additive versus antagonistic cytotoxic effects of the combined treatment of cells with masitinib and mitoxantrone were assessed by the combination index (CI) where CI<1, CI=1 and CI>1 indicate synergism, additive and antagonism, respectively.

### Results

After 24, 48 and 72 hours of treatment, the determination of cell number showed that mitoxantrone inhibited cell growth in a time- and dose-dependent manner. After 72 hours incubation the mitoxantrone IC₅₀ for CMT-U27 and CMT-U309 were 0.22 and 0.75 μM, respectively. Conversely, masitinib showed weak inhibition of these cell lines (IC₅₀ of 7.5 and 8.5 μM, respectively). Summary of the results is presented in Table 5.

The concentration of agents for use in the combination assays were based on their 72 hour single-agent IC₅₀ data. The combination of masitinib and mitoxantrone produced strong synergistic growth inhibition of both CMT-U27 and CMT-U309 cell lines with CIs of 0.549, and 0.809, respectively. Summary of the results is presented in Table 6.

These results show that masitinib mesilate strongly enhances the sensitivity of these cell lines to mitoxantrone. Taken together with those data showing masitinib mesilate sensitizes breast cancer cells to doxorubicin these results, surprisingly, show that masitinib mesilate is synergistic with anthracycline agents in vitro for the inhibition of breast cancer cell lines.

TABLE 5
Growth inhibitory activity of masitinib and
mitoxantrone on CMT-U27 and CMT-U309 cells

| Cell line | Treatment | Time | IC₅₀ (μM) |
|---|---|---|---|
| CMT-U27 | Masitinib | 24 h | 9.129 |
| | | 48 h | 7.607 |
| | | 72 h | 7.498 |
| CMT-U27 | Mixoxantrone | 24 h | 3.091 |
| | | 48 h | 1.108 |
| | | 72 h | 0.219 |
| CMT-U309 | Masitinib | 24 h | 15.032 |
| | | 48 h | 8.871 |
| | | 72 h | 8.545 |
| CMT-U309 | Mitoxantrone | 24 h | 2.774 |
| | | 48 h | 1.00 |
| | | 72 h | 0.751 |

TABLE 6
Growth inhibitory activity of masitinib and mitoxantrone
after 72 hours incubation and combination index (CI) values
of the masitinib plus mitoxantrone combination in CMT-U27 and
CMT-U309 cell lines. Masitinib and mitoxantrone doses at single-
agent IC₅₀ concentrations

| | | Viability | | Interpre- |
|---|---|---|---|---|
| Cell line | Treatment | (%) | CI | tation |
| CMT-U27 | Masitinib alone | 77.3 | | |
| | Mixoxantrone alone | 42.0 | | |
| | Masitinib + mixoxantrone | 28.2 | 0.549 | Synergism |
| CMT-U309 | Masitinib alone | 66.0 | | |
| | Mixoxantrone alone | 56.8 | | |
| | Masitinib + mixoxantrone | 42.7 | 0.809 | Synergism |

### Example 3: Masitinib but not Imatinib or Dasatinib can Resensitize Gemcitabine Resistant Cancer Cell Lines

As shown in Examples 1 and 2, masitinib can reverse resistance to chemotherapy and generate synergistic growth inhibition in various human and canine breast cancer cell lines. The potential of masitinib over other tyrosine kinase inhibitors (TKI) such as imatinib and dasatinib to enhance gemcitabine cytotoxicity was also investigated. Findings showed a surprising and unexpected synergy by the combination of masitinib with gemcitabine over the combination of other known c-Kit inhibitors such as imatinib or dasatinib with gemcitabine for treating solid tumors. Specifically, it was found that only masitinib is able to restore sensitivity to gemcitabine in Mia Paca-2-cells.

Imatinib (Gleevec, STI-571; Novartis) is a TKI targeting ABL, PDGFR, and c-Kit. Dasatinib (Sprycel, Bristol-Myers Squibb) is a TKI targeting SRC, ABL, PDGFR, and c-Kit. The structures of imatinib and dasatinib appear below:

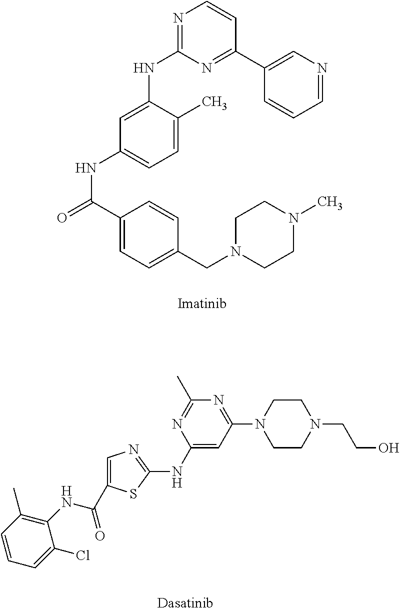 [SMILES: Cc1ccc(NC(=O)c2ccc(CN3CCN(C)CC3)cc2)cc1Nc1nccc(-c2cccnc2)n1 ; Cc1nc(Nc2ncc(C(=O)Nc3c(C)cccc3Cl)s2)cc(N2CCN(CCO)CC2)n1]

The potential of masitinib, imatinib, or dasatinib to enhance gemcitabine cytotoxicity is assessed by pretreating gemcitabine-resistant cell lines (Mia Paca-2) with these tyrosine kinase inhibitors (TKI) overnight and then exposing them to different doses of gemcitabine to record the IC₅₀ concentrations.

### Methodology

The antiproliferative activity of each TKI and gemcitabine is assessed using a WST-1 proliferation/survival assay in growth medium containing 1% fetal calf serum (FCS). Treatment is started with the addition of the relevant drug. For combination treatment (TKI plus gemcitabine), cells are re-suspended in medium (1% FCS) containing 0, 5 or 10 μM of the relevant TKI and incubated overnight before gemcitabine addition. After 72 hours, WST-1 reagent is added and incubated with the cells for 4 hours before absorbance measurement at 450 nm in an EL800 Universal Microplate Reader (Bio-Tek Instruments Inc.). Media alone is used as a blank and proliferation in the absence of drug served as a positive control. A TKI sensitization index is calculated as the ratio of the IC₅₀ of gemcitabine against the IC₅₀ of the drug combination.

### Results

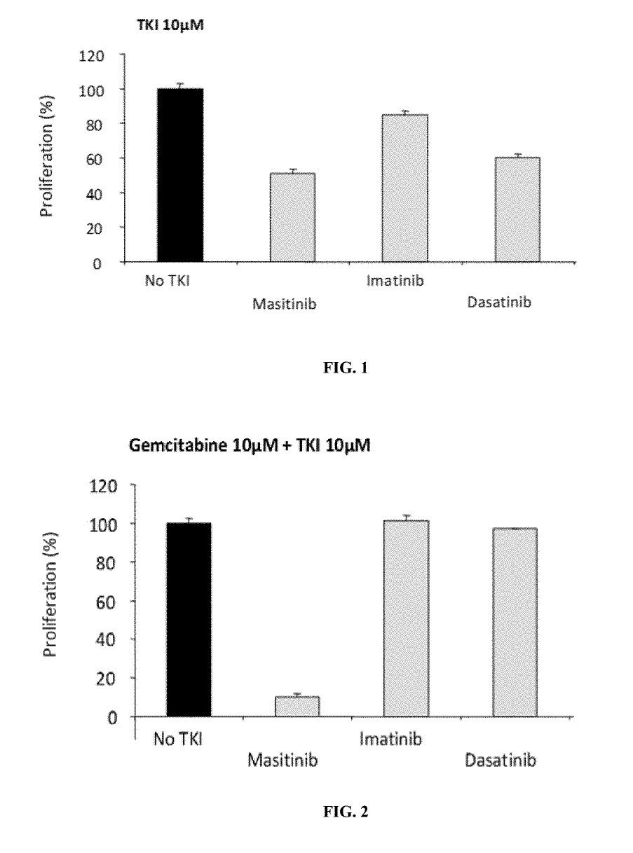

The results for the sensitivity of resistant Mia Paca-2 cells to various single agent treatment confirmed that these cells exhibit resistance (IC₅₀ superior to 10 μM) to gemcitabine. Masitinib alone does not significantly affect the growth of Mia Paca-2 cells (IC₅₀ of 5 to 10 μM), with more than 50% of the cells remaining resistant at a masitinib concentration of 10 μM (FIG. 1). Mia Paca-2 cell proliferation is affected by a similar extent when exposed to dasatinib (10 μM) as a single agent (FIG. 1).

However, a partial inhibition in the presence of low concentrations (superior to 0.1 μM) is also observed for this TKI. Mia Paca-2 cell proliferation is only weakly inhibited by imatinib (10 μM) as a single agent (FIG. 1).

Considering the sensitivity of Mia Paca-2 cells to the combination treatment of gemcitabine plus a given TKI, it is revealed that Mia Paca-2 cells are significantly sensitized with masitinib at 5 and 10 μM, as evidenced by the substantial reductions in cell proliferation (FIG. 2) and in gemcitabine IC₅₀. Specifically, the IC₅₀ of gemcitabine as a single agent is superior to 10 μM compared with an IC₅₀ of 0.025 μM for the masitinib (10 μM) plus gemcitabine combination, producing a sensitization factor superior to 400.

This antiproliferative action is also confirmed via microscopic observation, which clearly revealed cells to be dying rather than being arrested in the cell cycle. Conversely, pre-incubation of cells with 10 μM of imatinib or dasatinib does not result in an increased response of Mia Paca-2 cells to gemcitabine as compared to masitinib (FIG. 2).

### Example 4: Case Reports Demonstrating that Masitinib is Active in the Treatment of Inflammatory Breast Cancer (IBC)

### Case 1

This patient was initially diagnosed with invasive ductal carcinoma, T2N0M0, TNM-based stage I, and Scarff-Bloom-Richardson (SBR) grade II. No nodes were involved out of 9 sampled nodes. Immuno-histochemistry revealed positive status for estrogen receptors (ER+), negative status for progesterone receptors (PR−), and negative HER2 status.

At screening for treatment with masitinib, the patient was in failure to first-line of treatment including taxane treatment with clinically classified metastatic inflammatory breast cancer (lung).

TABLE 7
Treatment of a patient suffering from metastatic
inflammatory breast cancer (Case 1)

| | |
|---|---|
| Diagnosis | Metastatic inflammatory breast cancer |
| Treatment | Masitinib (6 mg/kg/day) administered |
| | orally in two daily doses plus |
| | gemcitabine (1250 mg/m² |
| | intravenous on days 1 and 8, |
| | over a 3-week cycle) |
| Masitinib exposure | 12 weeks |
| Best response (Modified- | Stable disease |
| RECIST criteria v1.1) | |
| Median overall survival | 20.4 months (deceased) |
| Age | 53 |
| Previous treatment failure | Taxane |
| Clinical classification at | Metastatic (lung) |
| screening (location) | |
| Histology at initial diagnosis | Invasive ductal carcinoma |
| Progesterone receptor (PR) | Negative |
| status | |
| Estrogen receptor (ER) status | Positive |
| HER2 status | Negative |

### Case 2

This patient was initially diagnosed with invasive ductal carcinoma, T2N0M0, TNM-based stage III, and Scarff-Bloom-Richardson (SBR) grade III. Eleven nodes were involved out of 14 sampled nodes. Immuno-histochemistry revealed positive status for estrogen receptors (ER+), negative status for progesterone receptors (PR−), and positive HER2 status (2+).

At screening for treatment with masitinib, the patient was in failure to first-line of treatment including taxane treatment with clinically classified metastatic inflammatory breast cancer (bone, mediastinal, and lung).

TABLE 8
Treatment of a patient suffering from metastatic
inflammatory breast cancer (Case 2)

| | |
|---|---|
| Diagnosis | Metastatic inflammatory breast cancer |
| Treatment | Masitinib (6 mg/kg/day) administered |
| | orally in two daily doses plus carboplatin |
| | (5AUC intravenous on day 1 over a 3- |
| | week cycle) |
| Dose reduction after week 3 | Masitinib (4.5 mg/kg/day) administered |
| | orally in two daily doses plus carboplatin |
| | (5AUC intravenous on day 1 over a 3- |
| | week cycle) |
| Masitinib exposure | 18 weeks |
| Best response (Modified- | Stable disease |
| RECIST criteria v1.1) | |
| Median overall survival | 20.9 months (deceased) |
| Age | 43 |
| Previous treatment failure | Taxane |
| Clinical classification at | Metastatic (bone, mediastinal, lung) |
| screening (location) | |
| Histology at initial diagnosis | Invasive ductal carcinoma |
| Progesterone receptor (PR) | Negative |
| status | |
| Estrogen receptor (ER) status | Positive |
| HER2 status | Positive |

Taken together, these case reports demonstrate that masitinib has a surprising and unexpected positive treatment-effect in refractory inflammatory breast cancer as compared with the benchmark of 11.2 months for lapatinib in this population (Kaufman B, et al. Lancet Oncol. 2009 June; 10(6):581-8).

### Example 5: Treatment of TNBC with a Combination of Masitinib and Cisplatin (Phase 1/2 Study)

A prospective, multicenter, open-label, uncontrolled, phase 1/2 clinical study has been conducted to evaluate efficacy and safety of masitinib mesilate in association with cisplatin after a first-line of cytotoxic chemotherapy of patients suffering from TNBC (either metastatic or locally advanced).

### Methodology

Sixteen TNBC patients resistant to at least one first line of chemotherapy including anthracycline or taxane were enrolled. In this open-label study, masitinib mesilate was administered orally at the daily dose of 9 mg/kg in two intakes, in combination with cisplatin given at the dose of 75 mg/m² by intravenous infusion every 3 weeks.

### Results

In the first phase 2 study, overall survival with masitinib mesilate in combination with cisplatin is 8.9 months and comparable to the existing standard survival in TNBC.

Overall survival (OS) is defined as the time from first treatment intake to the date of documented death. If death was not observed, data on OS were censored at the last date patient was known to be alive. OS was analyzed using Kaplan-Meier and was given with its confidence interval (CI) of 95%.

The median OS for patients with already one previous chemotherapy line was 10 months with a 95% CI [6.8-16.0]. In comparison, the treatment with cisplatin alone leads to a median OS of 9.4 (Baselga J, et al: J Clin Oncol 31:2586-2592, 2013)). Moreover, Kaplan Meier analysis of OS performed on patients with more than two previous chemotherapy lines showed that patients already treated with more than two previous chemotherapy lines had a higher OS than patients with less than 2 previous chemotherapy lines (median overall survival were: 13 versus 10.0 months). Summary of the results is presented in Table 9.

TABLE 9
Overall Survival

| | Median OS | |
|---|---|---|
| | (months) | [95% CI] |
| Cisplatin (N = 58) | 9.4 | |
| Masitinib plus cisplatin: | | |
| 1 previous chemotherapy line (N = 8) | 10 | [7.2; 19] |
| >2 previous chemotherapy lines (N = 4) | 13 | [6.8; 16] |

In conclusion, patients with metastatic and locally advanced TNBC and treated with the combination of masitinib mesilate plus cisplatin showed an improved efficacy compared to single-agent cisplatin when they had received at least one previous chemotherapy line.

### Example 6: Treatment of TNBC with a Combination of Masitinib, Gemcitabine and Carboplatin (Phase 1/2 Study)

The objective of this prospective, multicenter, central allocation, open-label, 3-parallel groups, phase 1/2 study is to evaluate efficacy and safety of masitinib mesilate at 6 or 9 mg/kg/day in association with gemcitabine plus carboplatin in patients with a triple negative breast cancer (either metastatic or locally advanced).

### Methodology

Twenty-one patients suffering from a metastatic or locally advanced triple-negative breast cancer have been enrolled. In this open-label study, masitinib mesilate was administered orally at a dose of 6 or 9 mg/kg/day in two intakes. The chemotherapeutic agents of gemcitabine and carboplatin were administered by intravenous injection, gemcitabine at a dose of 750 or 1000 mg/m² at day 1 and 8 of a cycle of 3 weeks and carboplatin at a dose of AUC 5 at day 1 of a cycle of 3 weeks.

### Preliminary Results

In the on-going phase 2 study, preliminary results show that current median overall survival on this study is 10.2 months for masitinib mesilate plus gemcitabine plus carboplatin. Results of OS are presented in Table 10.

TABLE 10
Overall Survival

| | Median OS | |
|---|---|---|
| | (months) | [95% CI] |
| Benchmark* | 6-7 | |
| Masitinib plus gemcitabine plus | 10.2 | [9.2-13.2] |
| carboplatin (N = 21) | | |

*O'Shaughnessy et al. N Engl J Med. 2011 Jan. 20; 364(3): 205-14. O'Shaughnessy J, et al. J Clin Oncol 27: 18s, 2009 (suppl; abstr 3)

The treatment with masitinib mesilate plus gemcitabine plus carboplatin increases the OS of patients suffering from triple negative metastatic or locally advanced breast cancer and compared favorably to the benchmark of gemcitabine plus carboplatin estimated from the BSI-201 study's 2009 interim analysis with minimal treatment cross-over (about 5.7 months) and final 2011 study results that would be confounded by treatment-arm crossover (about 7.7 months).

### Example 7: Treatment of Metastatic or Locally Advanced Breast Cancer with a Combination of Masitinib and Capecitabine or Gemcitabine (Phase 1/2 Study)

The objective of this prospective, multicenter, open-label, randomized, uncontrolled, 3-parallel groups, phase 1/2 study was to evaluate efficacy and safety of masitinib mesilate at 6 or 9 mg/kg/day in association with capecitabine or gemcitabine in patients with a metastatic or locally advanced breast cancer (all hormonal status except triple negative tumor) except with a triple negative tumor.

### Methodology

Twenty-seven patients suffering from metastatic or locally advanced breast cancer (with all hormonal status tumor except triple negative tumor) and resistant to at least one first line of chemotherapy including anthracycline or taxane have been enrolled for treatment with the combinations of masitinib plus gemcitabine, or masitinib plus capecitabine. In this open-label study, masitinib mesilate was administered orally at the daily dose of 6 or 9 mg/kg in two intakes, in combination with capecitabine orally administered at the dose of 1250 mg/m² twice daily for 2 weeks followed by a 7-day rest period (in cycles of 3 weeks) or with gemcitabine infused at the dose of 1000 or 1250 mg/m² at days 1 and 8 of a cycle of 3 weeks.

### Results

Preliminary results are presented in the Table 11 below. The data of the literature indicate that the median OS for patients resistant to at least one first-line of chemotherapy is 14.5 months with single-agent capecitabine and about 12.6 months for single-agent gemcitabine.

TABLE 11
Overall Survival

| | Median OS | |
|---|---|---|
| | (months) | [95% CI] |
| Benchmark for single-agent | 14.5 | — |
| capecitabine (L2)* | | |
| Benchmark for single-agent | 12.6 | [3.9-30.8] |
| gemcitabine (L2) ** | | |
| Masitinib plus gemcitabine (N = 13) | 20.9 | [19.9-NR] |
| Masitinib plus capecitabine (N = 14) | 22.5 | [13.7-NR] |

*Miller et al. J Clin Oncol 23 (4): 792-9, 2005.
** Brodowicz et al. Breast. 2000 December; 9(6): 338-42.

These preliminary results show that the treatment of patients suffering from metastatic or locally advanced breast cancer with a combination of masitinib mesilate and capecitabine or gemcitabine increases their survival.

1-27. (canceled)

28. A method for treating inflammatory breast cancer (IBC) in a subject in need thereof, comprising administering to said subject a therapeutically effective amount of masitinib or a pharmaceutically acceptable salt or solvate thereof in combination with a therapeutically effective amount of at least one chemotherapeutic agent.

29. The method according to claim 28, wherein the pharmaceutically acceptable salt or solvate of masitinib is masitinib mesilate.

30. The method according to claim 28, for improving survival and/or life expectancy of the subject.

31. The method according to claim 28, comprising sensitizing to a chemotherapeutic agent or restoring sensitivity to chemotherapy in the subject.

32. The method according to claim 28, wherein inflammatory breast cancer (IBC) is advanced IBC, locally advanced IBC or metastatic IBC.

33. The method according to claim 28, wherein inflammatory breast cancer is triple-negative inflammatory breast cancer.

34. The method according to of claim 28, wherein inflammatory breast cancer (IBC) is relapsed IBC or is refractory IBC.

35. The method according to claim 28, wherein the subject is naïve to anti-breast cancer treatments, or wherein inflammatory breast cancer relapsed after at least one anti-breast cancer treatment, or after two or more anti-breast cancer treatments.

36. The method according to claim 35, wherein anti-breast cancer treatments are selected from the group consisting of treatment with one or more chemotherapeutic agent, surgery, radiotherapy and any combination thereof.

37. The method according to claim 28, wherein said at least one chemotherapeutic agent is selected from the group consisting of: abitrexate (Methotrexate), abraxane (Paclitaxel Albumin-stabilized Nanoparticle Formulation), ado-Trastuzumab Emtansine, adrucil (Fluorouracil), afinitor (Everolimus), anastrozole, aredia (Pamidronate Disodium), arimidex (Anastrozole), aromasin (Exemestane), carboplatin, capecitabine, cisplatin, Clafen (Cyclophosphamide), Cyclophosphamide, Cytoxan (Cyclophosphamide), Docetaxel, Doxorubicin Hydrochloride, Efudex (Fluorouracil), Ellence (Epirubicin Hydrochloride), Epirubicin Hydrochloride, Eribulin Mesylate, Everolimus, Exemestane, Fareston (Toremifene), Faslodex (Fulvestrant), Femara (Letrozole), Fluoroplex (Fluorouracil), Fluorouracil, Folex (Methotrexate), Folex PFS (Methotrexate), Fulvestrant, Gemcitabine Hydrochloride, Gemzar (Gemcitabine Hydrochloride), Goserelin Acetate, Halaven (Eribulin Mesylate), Herceptin (Trastuzumab), Ixabepilone, Ixempra (Ixabepilone), Kadcyla (Ado-Trastuzumab Emtansine), Lapatinib Ditosylate, Letrozole, Megace (Megestrol Acetate), Megestrol Acetate, Methotrexate, Methotrexate LPF (Methotrexate), Mexate (Methotrexate), Mexate-AQ (Methotrexate), mitoxantrone, Neosar (Cyclophosphamide), Nolvadex (Tamoxifen Citrate), Paclitaxel, Paclitaxel Albumin-stabilized Nanoparticle Formulation, Pamidronate Disodium, Perjeta (Pertuzumab), Pertuzumab, Tamoxifen Citrate, Taxol (Paclitaxel), Taxotere (Docetaxel), Trastuzumab, Toremifene, Tykerb (Lapatinib Ditosylate), Velban (Vinblastine Sulfate), Velsar (Vinblastine Sulfate), Vinblastine Sulfate, Xeloda (Capecitabine), Zoladex (Goserelin Acetate), and Irinotecan.

38. The method according to claim 28, wherein said at least one chemotherapeutic agent is selected from anthracyclines, taxanes, platinum based chemotherapeutic agents, antimetabolites, and mixtures thereof.

39. The method according to claim 28, wherein said at least one chemotherapeutic agent is selected from anthracyclines.

40. The method according to claim 39, wherein said anthracycline is selected from the group consisting of doxorubicin, epirubicin, mitoxantrone, pixantrone, losoxantrone and daunorubicin, and any mixtures thereof.

41. The method according to claim 28, wherein said at least one chemotherapeutic agent is selected from the group consisting of gemcitabine, capecitabine, cisplatin, carboplatin, doxorubicin, mitoxantrone and any mixtures thereof.

42. The method according to claim 28, wherein the therapeutically effective amount of masitinib or a pharmaceutically acceptable salt or solvate thereof ranges from about 6 mg/kg/day to about 9 mg/kg/day.

43. The method according to claim 28, wherein masitinib or a pharmaceutically acceptable salt or solvate thereof is orally administered.

44. A method for inhibiting tyrosine kinases selected from the group consisting of c-Kit, LYN, FYN and PDGFR α and β and for inducing an anti-tumoral Th1 immune response, in an inflammatory breast cancer patient, thereby treating inflammatory breast cancer, wherein said method comprises administering a therapeutically effective amount of masitinib or a pharmaceutically acceptable salt or solvate thereof in combination with a therapeutically effective amount of a chemotherapeutic agent.
